(19) United States
(12) Patent Application Publication
(10) Pub. No.: US 2022/0354974 A1
(43) Pub. Date: Nov. 10, 2022

(54) REAGENTS AND METHODS FOR LABELING MOLECULES WITH ASTATINE ISOTOPES IN HIGHER OXIDATION STATES

(71) Applicant: University of Washington, Seattle, WA (US)

(72) Inventors: D. Scott Wilbur, Seattle, WA (US); Ming-Kuan Chyan, Seattle, WA (US); Donald K. Hamlin, Seattle, WA (US)

(73) Assignee: University of Washington, Seattle, WA (US)

(21) Appl. No.: 17/730,130

(22) Filed: Apr. 26, 2022

Related U.S. Application Data
(60) Provisional application No. 63/181,126, filed on Apr. 28, 2021.

(51) Int. Cl. A61K 51/10; C07F 5/04

(52) U.S. Cl. CPC A61K 51/10; C07F 5/04; C07B 2200/05

(57) ABSTRACT

Reagents and methods for labeling biomolecules with astatine isotopes in higher oxidation states. The reagents and methods allow for efficient labeling of biomolecules, such as antibodies, without the formation of high molecular weight by products that arise due to ²¹¹At-promoted dimerization and higher aggregation of the conjugated biomolecules, and diminish cellular retention of astatine caused by cellular oxidization of the bonded astatine atom in vivo.

### CROSS-REFERENCE TO RELATED APPLICATION

This application claims the benefit of U.S. Application No. 63/181,126, filed Apr. 28, 2021, expressly incorporated herein by reference in its entirety.

### STATEMENT OF GOVERNMENT LICENSE RIGHTS

This invention was made with government support under Grant No. CA113431 awarded by the National Institutes of Health, and Grant No. DE-SC0018013 awarded by the US Department of Energy. The government has certain rights in the invention.

### BACKGROUND

Isotopes of the element astatine are of interest for use in therapy and imaging of human diseases, such as cancer, bacterial infections and viral infections. For example, the astatine isotope, astatine-211, decays by alpha particle emission, which can be used when targeted by molecular entities to bind with diseased or bacterial cells and kill those cells. A particularly important application of this concept is use of astatinated antibodies, antibody fragments, and peptides for targeting and killing cancer cells in the body. Also, photons emitted from the astatine isotope, astatine-209, can be used to image sites of disease and effect of therapy of on the disease in the human body.

Astatine, like other halogens in group 17 of the periodic chart, can undergo electrophilic and nucleophilic reactions to bond it to disease-targeting molecules. However, unlike other halogens, labeling of molecules with astatine isotopes has been particularly difficult. Nucleophilic substitution of astatine often requires harsh reaction conditions and can result in products that are not stable in vitro or in vivo. Electrophilic substitution on electron-rich moieties within molecules can be accomplished directly, but astatine-labeled electron-rich molecules are not generally stable to deastatination in vitro or in vivo. To circumvent the instability of astatine-labeled molecules, particularly in vivo instability, methods for labeling “non-activated”, or electron deficient aromatic carbon-based moieties have been developed. Unfortunately, despite the fact that astatine bonded to a non-activated aromatic compound is stable in vitro, it can be quite unstable to in vivo deastatination, which renders its use in therapy of human diseases problematic due to toxicity of released astatine atoms in non-targeted tissues. The in vivo instability of astatine labeled aromatic ring compounds has been shown to be caused by oxidation of the molecule-bonded astatine atom by oxidases found in cells.

The in vivo release of astatine by oxidation can be precluded by the use of boron-containing aromatic moieties, wherein the astatine atom bonded to a boron atom in that moiety is stronger than a carbon-astatine bond. A number of aromatic boron-containing reagents have been developed which are stable to in vivo deastatination. In vivo deastatination is readily observed because free astatine is localized in thyroid, stomach, lung and spleen. A particularly valuable measure of the release of astatine in vivo is to conduct “dual label” experiments, wherein the same molecule is labeled with radioiodine and an astatine isotope, then injected into an animal. In general, release of astatine from the molecule is observed if the concentration of astatine in stomach, thyroid (neck), lung and spleen is higher than that of the radioiodine in those tissues.

While the release of astatine in vivo can be circumvented by employing aromatic boron-astatine bonds, problematic in vitro and in vivo characteristics of astatinated molecules containing anionic boron cage moieties have been observed. One observation is that antibody dimers or aggregates are obtained when an oxidant (e.g., chloramine-T (ChT)) is used for direct astatination of antibodies conjugated with a boron cage moiety. Another observation is that astatine-containing molecules, or their metabolites, can be retained in tissues whereas the same molecule labeled with radioiodine is released from those tissues.

Despite the advances in the development of astatine labeling and the preparation of astatine-labeled antibodies and smaller biomolecules, a need exists for improved labeling reagents and methods that address the disadvantages noted above. The present disclosure seeks to fulfill this need and provides further related advantages.

### SUMMARY

In one aspect, the disclosure provides compounds useful for reaction with astatine to provide astatine-containing compounds and for conjugation to targeting agents. In certain embodiments, the compounds are represented by Formula (I):

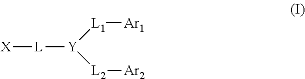

wherein

X is a group effective for associating the compound to a targeting agent;

L is a linker group that covalently links X to Y;

Y is trifunctional group that covalently links L, L₁, and L₂;

L₁ is a linker group that covalently links Y to Ar₁;

L₂ is a linker group that covalently links Y to Ar₂;

Ar₁ is an aromatic group that is reactive with an electropositive astatine species or is substituted with a group that makes it reactive with an electropositive astatine species and

Ar₂ is an aromatic group that is reactive with an electropositive astatine species or is substituted with a group that makes it reactive with an electropositive astatine species.

In another aspect, the disclosure provides astatine-containing compounds. In certain of these embodiments, the compound is represented by Formulae (IIA)-(IID):

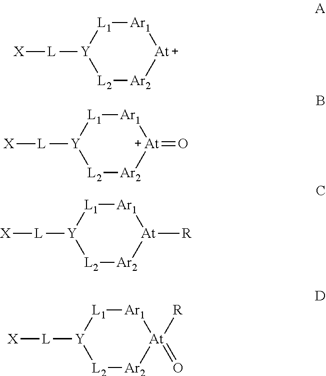

wherein X, L, Y, L₁, Ar₁, L₂, and Ar₂ are as defined above for the compounds and R is a halide selected from Cl, Br, and I, or hydroxyl, thiocyanate, isothiocyanate, or sulfhydryl, and for structures A and C the astatine atom is in the +3 oxidation state and for structures B and D the astatine atom is in the +5 oxidation state.

In another aspect, the disclosure provides conjugates that target the delivery of astatine. In certain embodiments, the conjugates are represented by Formula (III):

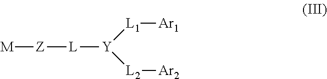

wherein

M is a targeting agent;

Z is a group formed from linking the targeting agent to L (via covalent coupling of group X of a compound as described herein with a suitably reactive group (e.g., —NH₂) on the targeting agent, or via binding of group X (ligand) of a compound as described herein with a suitably binding partner on the targeting agent);

L is a linker group that covalently links Z to Y;

Y is trifunctional group that covalently links L, L₁, and L₂;

L₁ is a linker group that covalently links Y to Ar₁;

L₂ is a linker group that covalently links Y to Ar₂;

Ar₁ is an aromatic group that is reactive with an electropositive astatine species or is substituted with a group that makes it reactive with an electropositive astatine atom; and

Ar₂ is an aromatic group that is reactive with an electropositive astatine species or is substituted with a group that makes it reactive with an electropositive astatine atom.

In other embodiments, the conjugate is an astatine-containing conjugate. In certain of these embodiments, the conjugate is represented by Formulae (IVE)-(IVH):

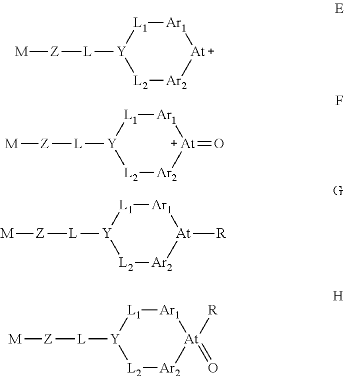

wherein M, Z, L, Y, L₁, Ar₁, L₂, and Ar₂ are as defined above, R is a halide (e.g., Cl, Br, and I), hydroxyl, thiocyanate, isothiocyanate, or sulfhydryl, and for structures E and G the astatine atom is in the +3 oxidation state and for structures F and H the astatine atom is in the +5 oxidation state.

In a further aspect, the disclosure provides methods for making an astatine compounds or astatine conjugates, methods for introducing an astatine isotope into a subject, and methods for treating a disease or condition treatable by the administration of an astatine isotope.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing aspects and many of the attendant advantages of this disclosure will become more readily appreciated as the same become better understood by reference to the following detailed description, when taken in conjunction with the accompanying drawings.

FIGS. 1A-1F compare size-exclusion chromatograms from reactions of a MAb-B10 (CA12.10C12-B10) conjugate with astatine using varying quantities (20, 10 and 5 μg) chloramine-T (ChT) (FIGS. 1A and 1B, 20 μg ChT; FIGS. 1C and 1D, 10 μg ChT; and FIGS. 1E and 1F, 5 μg ChT). FIGS. 1A, 1C, and 1E are radiochromatograms and FIGS. 1B, 1D, and 1F are chromatograms using UV detection (280 nm).

FIGS. 2A-2D compare size-exclusion chromatograms from reactions of a MAb-B10 (BC8-B10) conjugate with astatine using 20 μg ChT or without ChT (FIGS. 2A and 2B, 20 μg ChT; and FIGS. 2C and 2D, no ChT). FIGS. 2A and 2C are radiochromatograms and FIGS. 2B and 2D are chromatograms using UV detection (280 nm).

FIG. 3 is a schematic illustration of a possible route to formation of a dimeric MAb-B10 conjugate via astatination and subsequent oxidation of the astatine atom with an electropositive chlorine atom. It is likely that the astatine atom in the product will take on a positive charge to help offset the negative charges on the closo-decaborate(2-) moieties. Note that the circles in the boron cage structure represent boron atoms and the hydrogen atoms attached to the boron atoms have been left off for simplicity.

FIGS. 4A and 4B are bar graphs that compare concentrations of ¹²⁵I (blue bars) and ²¹¹At (red bars) in selected tissues when labeled using the non-cleavable pendant groups shown in compound. In FIG. 4A the radiolabeled B10 moiety is linked to the Fab′ through a lysine residue and in FIG. 4B the radiolabeled B10 moiety is linked through a cysteine moiety.

FIGS. 5A and 5B are bar graphs showing concentrations of ¹²⁵I (blue bars) and ²¹¹At (red bars) in selected tissues when labeled using the cleavable (hydrazone-containing) pendant group. The co-injected ¹²⁵I and ²¹¹At-labeled Fab′ conjugate (FIG. 5A) was developed to release the radiolabeled benzoic acid-B10 adduct (FIG. 5B).

FIGS. 6A-6E compare compound structure (6A) and radio-HPLC chromatograms showing products obtained in the oxidation of iodinated (6B, 6D) and astatinated (6C, 6E) electron deficient aryl compounds (polyethylene glycol linker used to increase aqueous solubility).

FIG. 7 compares concentrations of ¹²⁵I and ²¹¹At in selected tissues of mice at 1, 4 and 24 hours post injection of compounds shown of radioactive peaks from HPLC.

FIG. 8 is a schematic illustration of a possible reaction sequence for formation of an ²¹¹At-labeled aromatic compound (Ar) wherein an astatine atom is initially in the +1 oxidation state (i.e., astatide) is converted to an astatinated product with either a +3 (left panel) or a +5 (right panel) formal charge after reaction with an oxidant such as chloramine-T (ChT). Because there is some question as to whether At⁺ or AtO⁺ is the reactive species, reactions of both are shown. Note that the top reaction arrow shows the overall reaction from starting material to product, whereas a possible reaction sequence is shown by the arrows down, across and up to the astatinated product with a higher oxidation state.

FIG. 9 is a schematic illustration of a possible reaction sequence for formation of an ²¹¹At-labeled aromatic system where ²¹¹At is in a formal +3 oxidation state. The arch indicates that the two aromatic rings are in a single molecule. M represents an organometallic intermediate, such as nBu₃Sn or Me₃Si or B(OH)₂. In this example the second aromatic ring is a phenol, which can react in an electrophilic reaction as shown. The reaction result in a zwitterionic ²¹¹At-labeled product.

FIG. 10 is a schematic illustration of astatine (3+) labeling where two aromatic moieties are bonded with a single astatine atom. When at least one of the aromatic ring is negatively charged, the astatine atom will be positively charged. If both of the aromatic rings have neutral charges, A third atom or molecule will be bonded to the astatine atom.

FIG. 11 is a schematic illustration of a representative pendant group containing two aryl moieties for use in astatination of proteins, peptides, nucleotides, and small molecules.

FIG. 12 is a schematic illustration of chemical reactions used to obtain an isothiocyanatophenyl-bisB10 compound, and its conjugation with an antibody and subsequent astatination.

FIGS. 13A-13D compare size-exclusion chromatograms from reactions of a MAb-bisB10 (CA12.10C12-bisB10) conjugate with astatine using 30 μg chloramine-T (13A and 13B) and of a MAb-B10 (CA12.10C12-B10) conjugate with astatine using 30 μg chloramine-T (13C and 13D). FIGS. 13A and 13C are radiochromatograms and FIGS. 13B and 13D are chromatograms using UV detection (280 nm). Note that the area of the earlier eluting peak for the MAb-bisB10 is 11.4% of total by radioactivity and 11.0% of total by UV detection, whereas the area of the earlier eluting peak for the MAb-B10 is 26.6% of total by radioactivity and 5.6%% of total by UV detection.

FIG. 14 is a schematic illustration of a synthesis of a biotin-sarcosine-aryl-bisB10 compound and its astatination to form an astatonium bridged compound that has ²¹¹At is in a formal +3 oxidation state. It should be noted that if chloramine-T is used as an oxidant in the astatination reaction, the final product shown will be obtained directly from the bis-B10 derivative.

FIG. 15 is a schematic illustration of changes in formal charges and lipophilicity on Aryl-bisB10 compounds containing an astatonium ion bridge. R is any molecule and can include a linking moiety (L1) as shown in FIG. 11 but is preferred to be a biological targeting agent for treatment of human diseases.

FIG. 16 is a schematic illustration of changes in formal charges and lipophilicity on Aryl-bisB10 compounds containing an astatonium bridge. R is any molecule but is preferred to be a biological targeting agent for treatment of human diseases.

FIG. 17 is a schematic illustration of the synthesis of a representative B10-containing conjugation reagent (Compound 5).

FIGS. 18A-18H compare size-exclusion HPLC chromatograms showing products from ²¹¹At- and ¹²⁵I-labeling of CA12.10C12-B10 (MAb-B10) using varying quantities of chloramine-T: FIGS. 18A and 18B, ¹²⁵I labeling with 20 μg ChT; and FIGS. 18C-18H, ²¹¹At-labeling with 20 μg ChT (18C, 18D), 10 μg ChT (18E, 18F), and 5 μg ChT (18G, 18H). FIGS. 18A, 18C, 18E, and 18G are radiochromatograms and FIGS. 18B, 18D, 18F, and 18H are chromatograms using UV detection (280 nm).

FIGS. 19A-19D compare size-exclusion HPLC chromatograms showing products from ²¹¹At-labeling of 8C8-B10 using 20 μg ChT (19A, 19B) or no ChT (19C, 19D). FIGS. 19A and 19C are radiochromatograms and FIGS. 19B and 19D are chromatograms using UV detection (280 nm).

FIG. 20 is a schematic illustration of the preparation of a representative astatinated compound. Reaction of Compound 1 with ²¹¹At in 5% HOAc/MeOH/H2O without an added oxidant proceeds in a stepwise manner by radio-HPLC analysis, presumably undergoing very rapid reaction with one B10(2-) moiety to form an astatinated Compound 2, then reacting more slowly (30 min) with the second B10(2-) moiety to form bis-B10 astatonium ion Compound 3.

FIG. 21 is a schematic illustration of the preparation of a representative biotin-sarcosine-bis-B10 reagent.

FIGS. 22A-22D compare radio-HPLC chromatograms of reaction product from ²¹¹At labeling of Compound 8 (see FIG. 21): 0.1 mg of Compound 8 was reacted with ²¹¹At in 5% HOAc in MeOH/H₂O without ChT at room temperature for 5 min (22A) and the reaction mixture sat at room temperature for about 1 h (22B); and 0.1 mg of Compound 8 was reacted with ²¹¹At in 5% HOAc in MeOH/H₂O with 10 μg of ChT at room temperature for 3 min. (22C) the reaction mixture after setting at room temperature for about 2 h (22D).

FIGS. 23A-23C compare radio-HPLC chromatograms of reaction product from ²¹¹At-labeling of Compound 8 (see FIG. 21): 0.1 mg of 8 was reacted with ²¹¹At in 5% HOAc in MeOH/H₂O at room temperature for approximately 5 min (23A), 100 min (23B) and 130 min (23C).

FIG. 24 is a radio-HPLC chromatogram of ²¹¹At-labeled compound, Compounds 10a or 10b (see FIG. 21), after HPLC purification. The purified was evaporated to dryness, re-dissolved in PBS, then analyzed by radio-HPLC.

FIG. 25 is a schematic illustration of the synthesis of a bis-B10-NCS reagent and its conjugation to a monoclonal antibody and radiolabeling with ²¹¹At.

FIG. 26 is an image of a IEF gel comparing pIs of unaltered, mono-B10 conjugate and bis-B10 conjugates to IEF standards.

FIGS. 27A and 27B compare size-exclusion HPLC chromatograms showing gamma detection (27A) and UV detection (27B) of the products from ²¹¹At-labeling of CA12.10C12-bis-B10. Note that the area of HMW species is essentially the same in the UV chromatogram as in the gamma chromatogram.

FIGS. 28A and 28B compare size-exclusion HPLC chromatograms showing gamma detection (28A) and UV detection (28B) of the products from ²¹¹At-labeling of CA12.10C12-B10. Note that the area of HMW species is much lower (about 5%) in the UV chromatogram than in the gamma chromatogram.

### DESCRIPTION

The present disclosure provides compounds useful for reaction with astatine to provide astatine-containing compounds and for conjugation to targeting agents (e.g., large and small molecular weight biomolecules) to provide astatine-containing targeting agents, methods for making the compounds the astatine-containing compounds and astatine-containing targeting agents, methods for making the compounds and targeting agents, and methods for using the compounds and targeting agents.

The present disclosure provides reagents and methods for labeling biomolecules with astatine isotopes in higher oxidation states. The reagents and methods allow for efficient labeling of biomolecules, such as antibodies, without the formation of high molecular weight by products that arise due to ²¹¹At-promoted dimerization and higher aggregation of the conjugated biomolecules, and also to diminish cellular retention of astatine caused by cellular oxidization of the bonded astatine atom in vivo.

In one aspect, the disclosure provides compounds useful for reaction with astatine to provide astatine-containing compounds and for conjugation to targeting agents.

In certain embodiments, the compounds are represented by Formula (I):

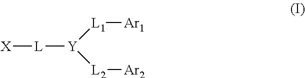

wherein

X is a group effective for associating the compound to a targeting agent;

L is a linker group that covalently links X to Y;

Y is trifunctional group that covalently links L, L₁, and L₂;

L₁ is a linker group that covalently links Y to Ar₁;

L₂ is a linker group that covalently links Y to Ar₂;

Ar₁ is an aromatic group that is reactive with an electropositive astatine species or is substituted with a group that makes it reactive with an electropositive astatine species and

Ar₂ is an aromatic group that is reactive with an electropositive astatine species or is substituted with a group that makes it reactive with an electropositive astatine species.

In the compounds described herein, electropositive astatine species may be coupled to the compound's aromatic groups (Ar₁ and Ar₂) (see compounds of Formula (I) and conjugates of Formula (III)) to provide astatine-bridged compounds (see compounds of Formulae (IIA)-(IID) and conjugates of Formulae (IVE)-(IVH)).

As used herein, the term “aromatic group” refers to a group of covalently bonded atoms (e.g., carbon and heteroatoms, such as boron, nitrogen, oxygen, sulfur) that includes a delocalized electrons in a conjugated π system (e.g., π bonds via p orbitals). The delocalized conjugated π system of electrons may result from a planar arrangement of atoms in a ring (e.g., phenyl, pyridyl) or rings (e.g., naphthyl) or a three-dimensional arrangement of atoms in a cluster (e.g., boron-caged compound, borate, carborane).

Suitable aromatic groups include carbocyclic aromatic groups (e.g., aryl groups), heterocyclic aromatic groups (e.g., heteroaryl groups), and aromatic boron groups (e.g., planar and three-dimensional boron clusters).

Suitable aryl groups include aromatic hydrocarbon groups having 6 to 10 carbon atoms. In some embodiments, the term “aryl” includes monocyclic or polycyclic (e.g., having 2 fused rings) aromatic hydrocarbons such as, for example, phenyl, naphthyl, anthracenyl, phenanthrenyl, indanyl, and indenyl. The aryl group may be further substituted with one or more substituents as described herein (e.g., optionally substituted with 1, 2, 3, or 4 substituents each independently selected from halo, hydroxyl, thiol, ether, thioether, amino, amido, C₁₋₆ alkyl, C₁₋₆ haloalkyl, and C₃-C₇ cycloalkyl). In certain embodiments, the aryl group is a phenol, a thiophenol, an aniline, an anisole, or an organometallic-substituted benzene.

Suitable heteroaryl groups include 5- to 10-membered aromatic monocyclic or bicyclic ring containing 1-4 heteroatoms selected from O, S, and N. Representative 5- or 6-membered aromatic monocyclic ring groups include pyridine, pyrimidine, pyridazine, furan, thiophene, thiazole, oxazole, and isooxazole. Representative 9- or 10-membered aromatic bicyclic ring groups include benzofuran, benzothiophene, indole, pyranopyrrole, benzopyran, quinoline, benzocyclohexyl, and naphthyridine. The heteroaryl group may be further substituted with one or more substituents as described herein (e.g., optionally substituted with 1, 2, 3, or 4 substituents each independently selected from halo, hydroxyl, thiol, ether, thioether, amino, amido, C₁₋₆ alkyl, C₁₋₆ haloalkyl, and C₃-C₇ cycloalkyl). In certain embodiments, the heteroaryl aryl group is a pyridine.

Suitable aromatic boron groups include anionic boron-caged compounds of varying sizes, including borates such as dodecaborates (e.g. closo-dodecaborate (2-), decaborates (e.g., closo-decaborate(2-)), and carboranes (e.g., monocarbon carboranes (R—CB₉H₉)), nido-carboranes).

As used herein, the term “electropositive astatine species” refers to an astatine atom in an oxidation state of +1 or an astatine-containing radical that includes an astatine atom in an oxidation state of +3 or +5. Representative astatine-containing radicals include At═O, At—R₂, and At(═O)R, wherein R is a halide (e.g., Cl, Br, I), hydroxyl, thiocyanate, isothiocyanate, or sulfhydryl.

In certain of these embodiments, X is a functional group that is effective for covalently coupling the compound to a targeting agent and is selected from amine- and sulfhydryl-reactive groups, such as active esters, isocyanates, isothiocyanates, and maleimides.

In other of these embodiments, X is a ligand of a binding partner pair that is effective for binding the compound to a targeting agent. Suitable binding pairs include ligands and their binding partners. Representative binding pairs include biotin and related derivatives and avidin or streptavidin, antigens and their corresponding antibodies or functional fragments thereof, and ligands and their receptors or functional fragments thereof.

In certain embodiments, the compound is a bis-B10 compound. In certain of these other embodiments, the compound is an aryl bis-B10 compound. In further of these embodiments, the compound is an isothiocyanatophenyl-bis-B10 compound. In a particular embodiment, the compound is a biotin-sarcosine-aryl-bis-B10 compound. As used herein, the term “B-10 compound” refers to compound that includes a closo-decaborate (2-) moiety. In certain embodiments, the compound includes two closo-decaborate (2-) moieties.

As noted above, in certain embodiments, the compound is an astatine-containing compound. In certain of these embodiments, the compound is represented by Formulae (IIA)-(IID):

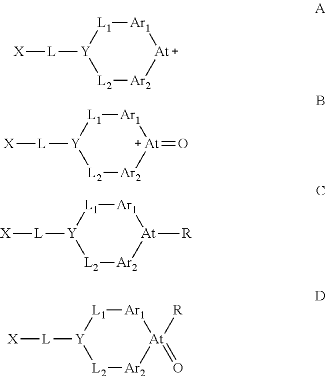

wherein X, L, Y, L₁, Ar₁, L₂, and Ar₂ are as defined above for the compounds and R is a halide selected from Cl, Br, and I, or hydroxyl, thiocyanate, isothiocyanate, or sulfhydryl, and for structures A and C the astatine atom is in the +3 oxidation state and for structures B and D the astatine atom is in the +5 oxidation state.

Astatine can be in −1, +1, +3, +5 and +7 oxidation state, but +7 state is rare. It should be noted that electropositive astatine species (shown in structures A and B) is a preferred embodiment as because of their increased stability in vivo. The electropositive At will most likely be present when either Ar₁ or Ar₂ are anionic, or both are anionic. Polarization of the At atom and/or oxidizing conditions are required to allow the At atom undergo electrophilic reaction with the second aryl group. However, for compounds that include the closo-decaborate (2-) moieties, it appears that no added oxidant is required. Without being bound to theory, it may be oxygen (O₂) or trace metals in solution or a combination of those elements that oxidize the bonded At.

The present disclosure provides compounds having two aromatic groups, each reactive with an electropositive astatine species. These compounds provide astatine-labeled compounds in which the astatine species bridges the aromatic groups. See, for example, compounds of Formulae (IIA)-(IID).

Similarly, in certain embodiments, the disclosure provides conjugates having two aromatic groups, each reactive with an electropositive astatine species. These conjugates provide astatine-labeled conjugates in which the astatine species bridges the aromatic groups. See, for example, conjugates of Formulae (IVA)-(IVD).

In another aspect, the disclosure provides conjugates that target the delivery of astatine.

In certain embodiments, the conjugates are represented by Formula (III):

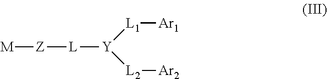

wherein

M is a targeting agent;

Z is a group formed from linking the targeting agent to L (via covalent coupling of group X of a compound as described herein with a suitably reactive group (e.g., —NH₂) on the targeting agent, or via binding of group X (ligand) of a compound as described herein with a suitably binding partner on the targeting agent);

L is a linker group that covalently links Z to Y;

Y is trifunctional group that covalently links L, L₁, and L₂;

L₁ is a linker group that covalently links Y to Ar₁;

L₂ is a linker group that covalently links Y to Ar₂;

Ar₁ is an aromatic group that is reactive with an electropositive astatine species or is substituted with a group that makes it reactive with an electropositive astatine atom; and

Ar₂ is an aromatic group that is reactive with an electropositive astatine species or is substituted with a group that makes it reactive with an electropositive astatine atom.

In other embodiments, the conjugate is an astatine-containing conjugate. In certain of these embodiments, the conjugate is represented by Formulae (IVE)-(IVH):

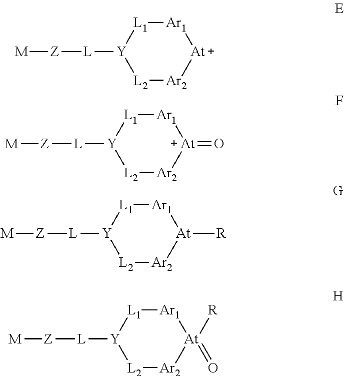

wherein M, Z, L, Y, L₁, Ar₁, L₂, and Ar₂ are as defined above, R is a halide (e.g., Cl, Br, and I), hydroxyl, thiocyanate, isothiocyanate, or sulfhydryl, and for structures E and G the astatine atom is in the +3 oxidation state and for structures F and H the astatine atom is in the +5 oxidation state.

In certain embodiments of the conjugate, M is a biomolecule. In certain of these embodiments, M is a cancer-targeting biomolecule. In certain embodiments, M is a small molecule (e.g., molecular weight less than 2000 g/mole) that is effective to target a site for delivery of astatine. In other embodiments, M is an antibody or a functional fragment thereof.

As used herein, the term “functional antibody fragment” (FAB) refers to proteins that form part of the antigen recognition site. FABs are produced in genetically modified bacteriophages, bacteria, fungi, or plants and, consequently, can be produced in large quantities at a fraction of the cost of traditional antibodies.

Antigen-binding fragments (Fab) and single chain variable fragments (scFV) are common antibody fragments that have been investigated and also another type known as “third generation” (3G) molecules. The Fab fragments are consisting of one constant and one variable domain of heavy and light chains, whereas in scFV fragments, the varying areas of heavy and light chains are merged and the constant heavy and light chain that was in the previous state is absent and in the case of the third type, it consists of only one variable heavy chain.

Suitable functional fragments include single chain variable fragments and third generation fragments.

In certain of the embodiments of the compounds and conjugates described above, L is adapted to provide a distance between X and Y, or Z and Y, respectively, and optionally comprises one or more functional groups (e.g., —O(CH₂CH₂O)—) to impart aqueous solubility and/or ionic charges to the compound or conjugate; L₁ is adapted to provide a distance between Y and Ar₁, and optionally comprises one or more functional groups to impart aqueous solubility (e.g., —O(CH₂CH₂O)—) and/or ionic charges to the compound or conjugate; and L₂ is adapted to provide a distance between Y and Ar₂, and optionally comprises one or more functional groups to impart aqueous solubility (e.g., —O(CH₂CH₂O)—) and/or ionic charges to the compound or conjugate.

In certain of these embodiments, L₁ and L₂ are the same. In other of these embodiments, L₁ and L₂ are different.

In certain of the embodiments of the compounds and conjugates described above, Y is a trifunctional aryl group (e.g., trifunctional phenyl group, such as 1,3,5-substituted phenyl group) or trifunctional alkyl group (e.g., amino acids, such as lysine, glutamic acid, modified serine).

In certain of these embodiments of the compounds and conjugates described above, Ar₁ and Ar₂ are independently anionic aromatic moieties or modified aromatic moieties that are reactive with an electropositive astatine species. In certain embodiments, Ar₁ and Ar₂ are independently selected from the group consisting of phenols, thiophenols, anilines, anisoles, organometallic substituted benzenes, nido-carboranes, monocarbon carboranes, and closo-borates (2-). In certain of these embodiments, Ar₁ and Ar₂ are the same. In other embodiments, Ar₁ and Ar₂ are different. In a particular embodiment, Ar₁ and Ar₂ are each closo-decaborate (2-).

In another aspect, the disclosure provides methods for making an astatine compounds or astatine conjugates.

In certain embodiments, the method comprises reacting an electropositive astatine species with a compound or conjugate having an aromatic group that is reactive toward the electropositive astatine species, wherein the reaction does not include the use of an oxidant.

In other embodiments, the method comprises reacting an electropositive astatine species with a compound or conjugate having an aromatic group that is reactive toward the electropositive astatine species, as described herein. In certain of these embodiments, the reaction does not include the use of an oxidant.

Representative compounds and conjugates include those described herein. Representative aromatic groups and electropositive astatine species include those described herein. In contrast to known labeling methods known in the art that utilize an oxidant (e.g., chloramine T), in certain embodiments, the method described herein advantageously does not include the use of an oxidant.

In another aspect of the disclosure, a method for introducing an astatine isotope into a subject is provided. In one embodiment, the method comprises administering an astatine-containing conjugate as described herein to a subject in need thereof.

In further aspect of the disclosure, a method for treating a disease or condition treatable by the administration of an astatine isotope is provided. In one embodiment, the method comprises administering a therapeutically effective amount of an astatine-containing conjugate as described herein to a subject in need thereof.

Methods for Labeling Molecules with Astatine Isotopes in Higher Oxidation States

This present disclosure addresses two important issues in the application of astatine isotopes to the treatment of human disease: (1) unwanted reaction side products being obtained when oxidants are used for radiolabeling molecules with astatine isotopes and (2) oxidation of astatinated molecules in cells (by oxidases) can cause the compounds to interact with cellular components, resulting in undesired retention in normal tissues such as kidney. These issues can be circumvented by predesigned labeling structures such that the astatine isotope is in a higher oxidation state (+3 or +5) in the radiolabeled molecule. By preparing compounds with astatine isotopes in a higher oxidation state the unwanted in vitro or in vivo oxidation of the isotope is precluded.

Isotopes of the element astatine are of interest for use in therapy and imaging of human diseases, such as cancer, bacterial infections and viral infections. For example, the astatine isotope, astatine-211, decays by alpha particle emission, which can be used when targeted by molecular entities to bind with diseased or bacterial cells and kill those cells. A particularly important application of this concept is use of astatinated antibodies, antibody fragments, and peptides for targeting and killing cancer cells in the body. Also, photons emitted from the astatine isotope, astatine-209, can be used to image sites of disease and effect of therapy of on the disease in the human body.

Astatine, like other halogens in group 17 of the periodic chart, can undergo electrophilic and nucleophilic reactions to bond it to disease-targeting molecules. However, unlike other halogens, labeling of molecules with astatine isotopes has been particularly difficult. Nucleophilic substitution of astatine often requires harsh reaction conditions and can result in products that are not stable in vitro or in vivo. Electrophilic substitution on electron-rich moieties on molecules can be accomplished directly, but astatine-labeled electron-rich molecules are not generally stable in vitro or in vivo. To circumvent the instability of astatine-labeled molecules, particularly in vivo instability, methods for labeling “non-activated”, or electron deficient aromatic carbon-based moieties have been developed. Unfortunately, despite the fact that astatine bonded to a non-activated aromatic compound is stable in vitro, it can be quite unstable to in vivo deastatination, which renders its use in therapy of human diseases problematic due to toxicity of released astatine atoms in non-targeted tissues. The in vivo instability of astatine labeled aromatic ring compounds has been shown to be caused by oxidation of the molecule-bonded astatine atom by oxidases found in cells.

The in vivo release of astatine by oxidation can be precluded by the use of boron-containing aromatic moieties, wherein the astatine atom bonded to a boron atom in that moiety is stronger than a carbon-astatine bond. A number of aromatic boron-containing reagents have been developed which are stable to in vivo deastatination. In vivo deastatination is readily observed because free astatine is localized in thyroid, stomach, lung and spleen. A particularly valuable measure of the release of astatine in vivo is to conduct “dual label” experiments, wherein the same molecule is labeled with radioiodine and an astatine isotope, then injected into an animal. In general, release of astatine from the molecule is observed if the concentration of astatine in stomach, thyroid (neck), lung and spleen is higher than that of the radioiodine in those tissues. While the release of astatine in vivo can be circumvented by employing aromatic boron-astatine bonds, problematic in vitro and in vivo characteristics of astatinated molecules containing boron cage moieties have been observed. One observation is that antibody dimers or aggregates are obtained when an oxidant, e.g., chloramine-T (ChT), is used for direct labeling of antibodies conjugated with a boron cage moiety. That dimer/aggregate is not observed if the labeling is done without an oxidant. Additionally, the dimer/aggregate is not observed when labeling the same antibody conjugate with iodine while using the oxidant ChT. This observation suggests that astatine can undergo oxidation post radiolabeling and that activated astatine atom can react with another antibody conjugate to cause dimerization.

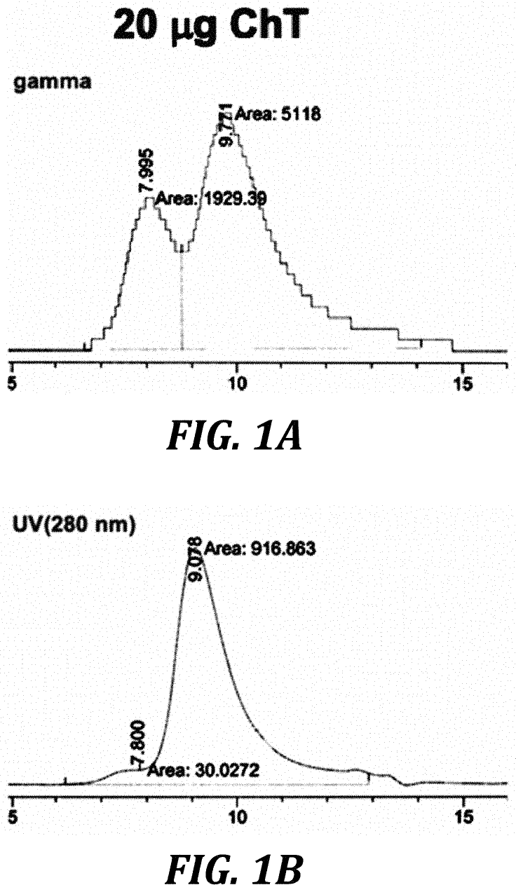

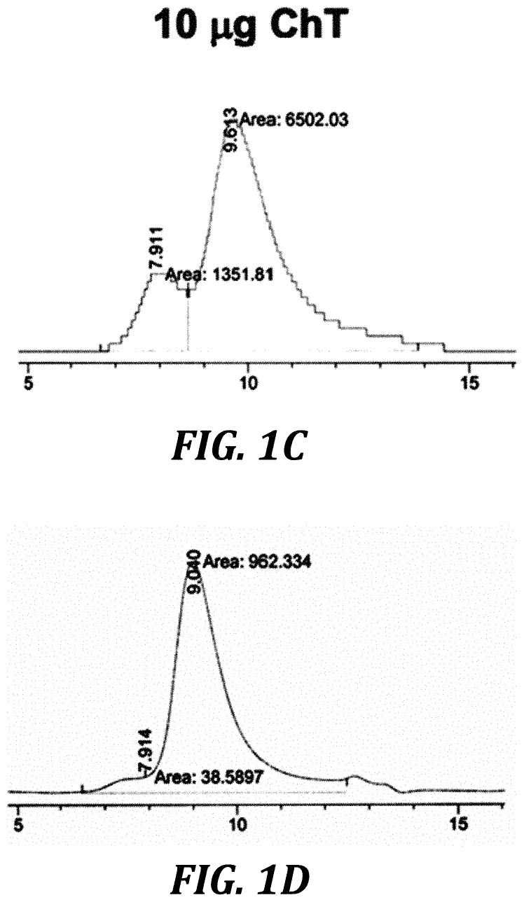

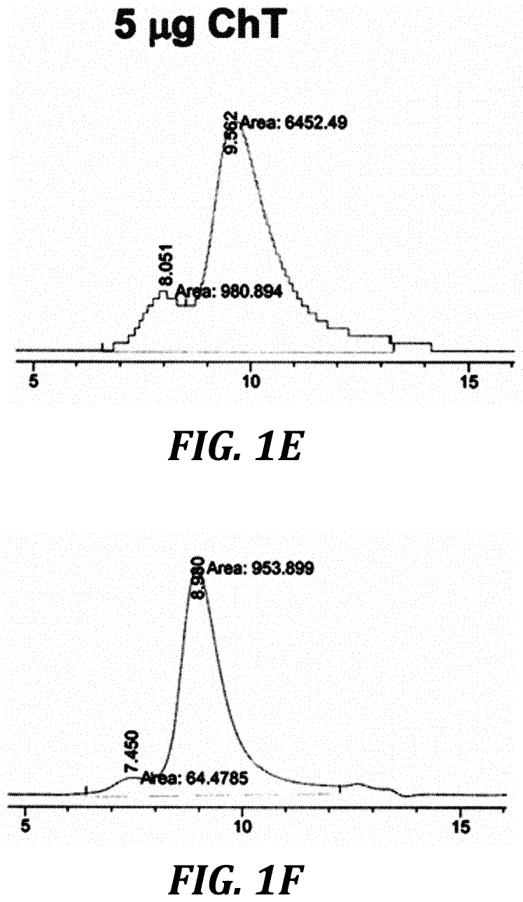

Astatine-labeling of monoclonal antibodies (MAb), conjugates containing the closo-decaborate (2-) moiety (referred to herein as MAb-B10) have been found to be particularly valuable as they can be directly labeled and provide high labeling yields. A large number of these reactions have been conducted over a period of several years. However, this reaction can have a major side reaction product when ChT is used to oxidize astatine. The true nature of that side product is not known, but it is believed to be a dimeric form of the MAb-B10 conjugate. Evidence that oxidation by ChT is the cause of the formation of a higher molecular weight species was obtained from astatine-labeling studies where a MAb-B10 (500 μg CA12.10C12-B10) was reacted with [²¹¹At]NaAt and decreasing quantities of ChT. The initial labeling studies were conducted with 20 μg of ChT (an amount generally used for radioiodinations). In subsequent labeling experiments the quantity of ChT was decreased to 10 μg, then decreased to 5 μg. After a reaction time of 1 minute, a reductant (sodium metabisulfite) was added to assure any free astatine was in the non-volatile sodium astatide form, then the reaction mixture was run over a Sephadex G-25 column (PD-10) for purification. Following purification, the MAb-B10-At was evaluated by radioHPLC. Size-exclusion HPLC chromatograms for this series of astatine labeling reactions are shown in FIGS. 1A-1F. The chromatograms are truncated to show protein (by UV) and radiolabeled protein (radioactivity detector) peaks more clearly. There were no other peaks on chromatograms.

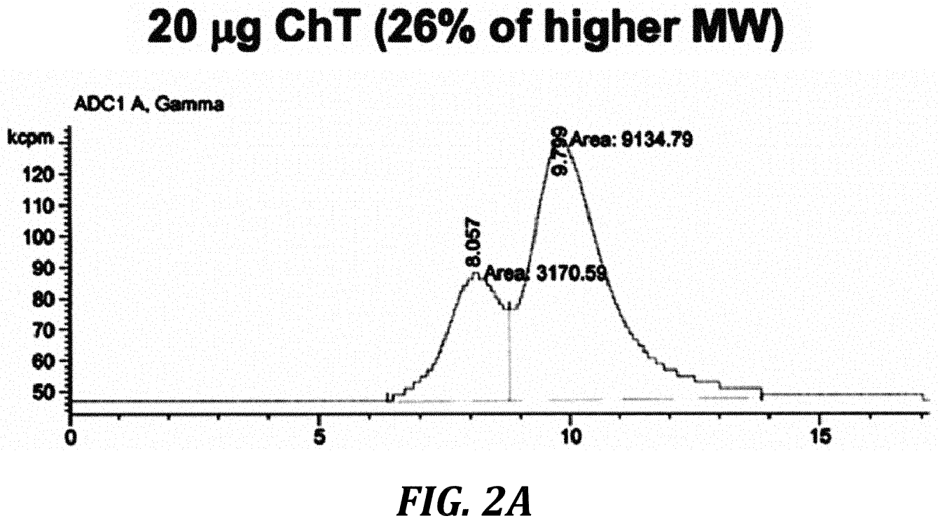

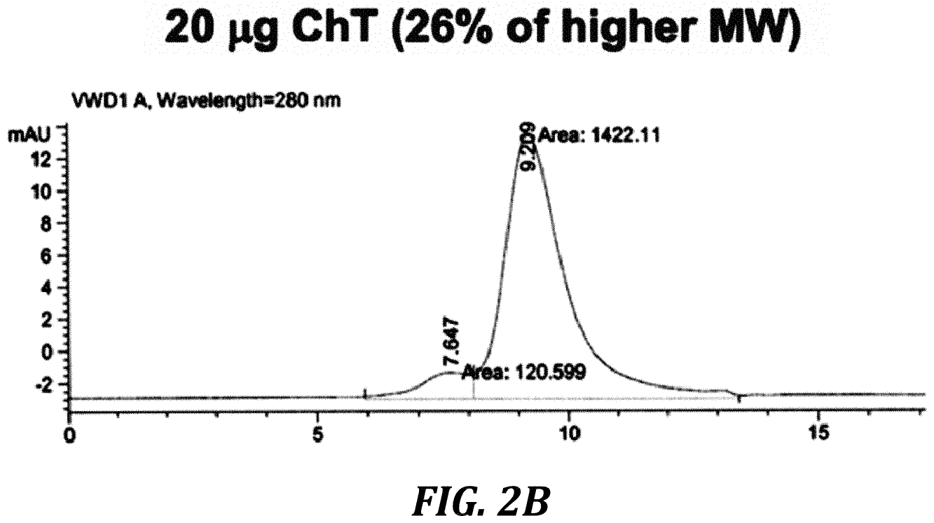

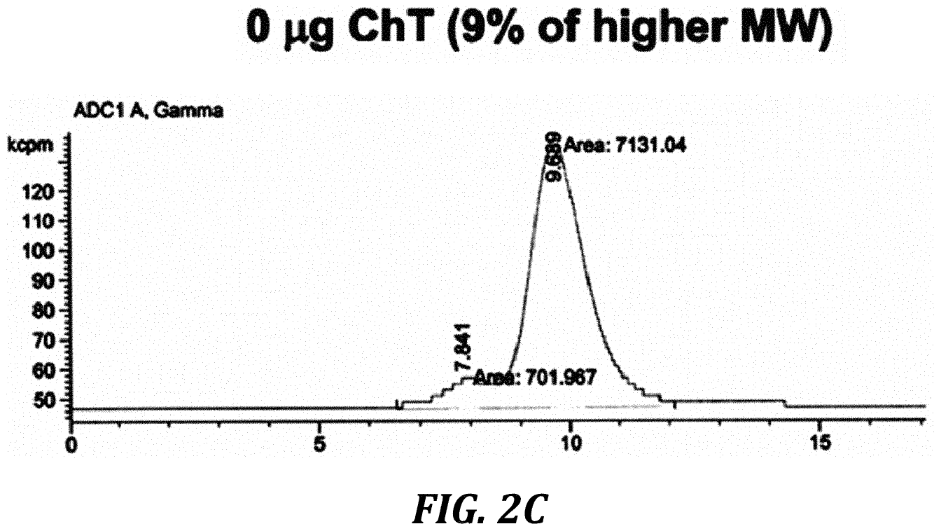

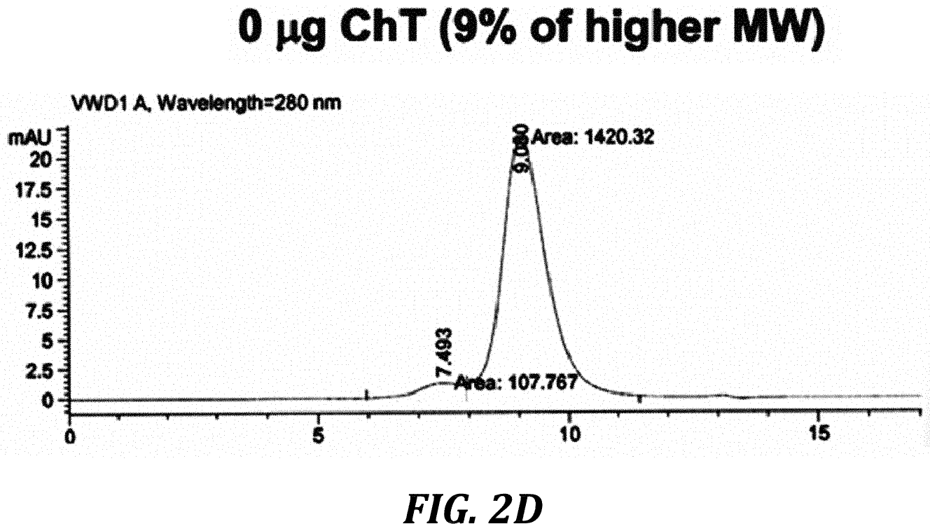

It is clear from the radiochromatograms in FIGS. 1A, 1C, and 1E that decreasing the amount of ChT in the reactions decreases the amount of higher molecular weight species. Also note that the protein peak, as detected by UV, does not show any (or very minor) change in the high molecular weight peak. Thus, the dimerization only occurs on the astatine-labeled antibody, indicating that only a very small amount of the MAb-B10 is involved. Further evidence for astatine-facilitated dimerization was observed when 500 g of another MAb-B10 (BC8-B10) was reacted with [²¹¹At]NaAt using 20 μg ChT and without ChT. As in the first example, the reaction was conducted for 1 min before quenching with sodium metabisulfite and purification over a PD-10 column. Chromatograms from that experiment are shown in FIGS. 2A-2D.

As in FIGS. 1A, 1C, and 1E, the radiochromatograms show that decreasing the amount of ChT in the reaction decreased the quantity of radiolabeled high molecular weight species. Indeed, without using any ChT oxidant, the amount of astatine-labeled high molecular weight species is essentially the same as the quantity of high molecular weight species present in the original antibody solution, suggesting no dimerization had occurred. These findings demonstrated that an oxidant was not required for electrophilic reactions to occur on the dianionic closo-decaborate(2-) species. This is likely due to air oxidation of the astatide, perhaps with trace metal catalysis. It clearly shows that use of oxidant caused an unwanted high molecular weight species to be formed. Again, it seems most likely that the high molecular weight species is a MAb-B10 dimer.

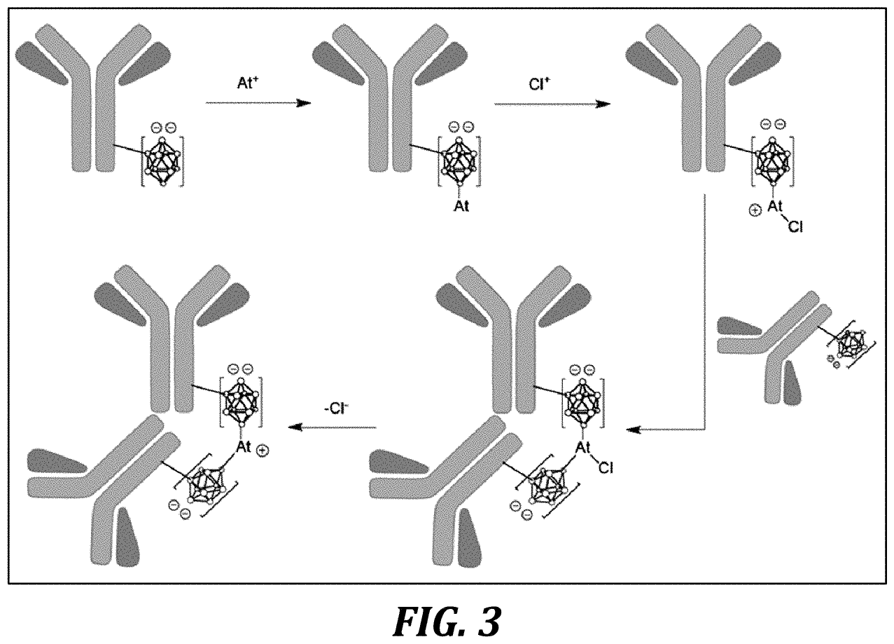

A general scheme depicting astatination of a MAb-B10 conjugate, wherein use of an oxidant (Cl⁺ from ChT) results in formation of a dimeric MAb-B10 compound with a cross-linking +3 astatine species, is shown in FIG. 3. This is similar to crosslinking of two anionic monocarbon carboranes with a positively charged +3 iodine bridge previously reported. Although it requires that two very large molecules containing closo-decaborate (2-) moieties arrange in such a manner as to result in MAb-B10 dimerization, as depicted in FIG. 3, the dimerization can be rationalized because of the high nucleophilicity of the dianionic closo-decaborate (2-) moiety. Also, if a quantity of compound containing the closo-decaborate (2-) moiety is mixed with the MAb-B10, no dimerization occurs. Further, radioiodination of the same MAb-B10 conjugate under the same reaction conditions does not result in dimer formation. While not proven, formation of a dimeric MAb-B10 bridged structure as shown in FIG. 3 best fits the data obtained.

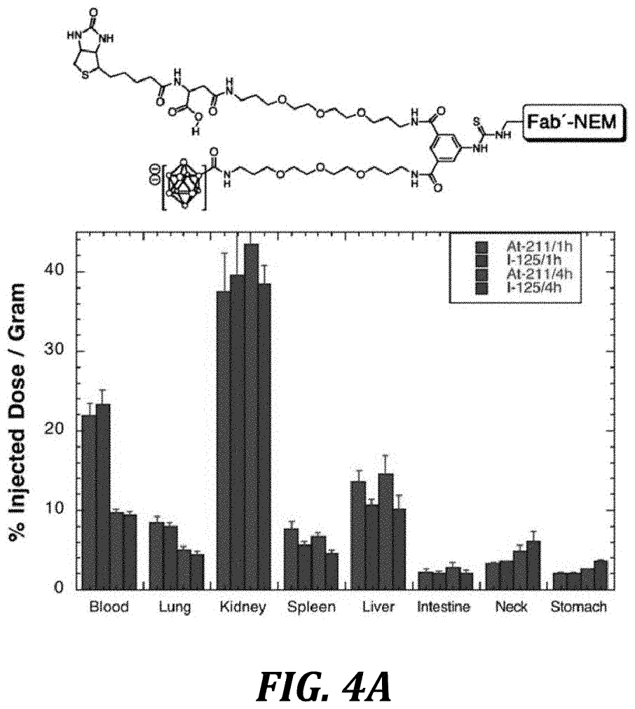

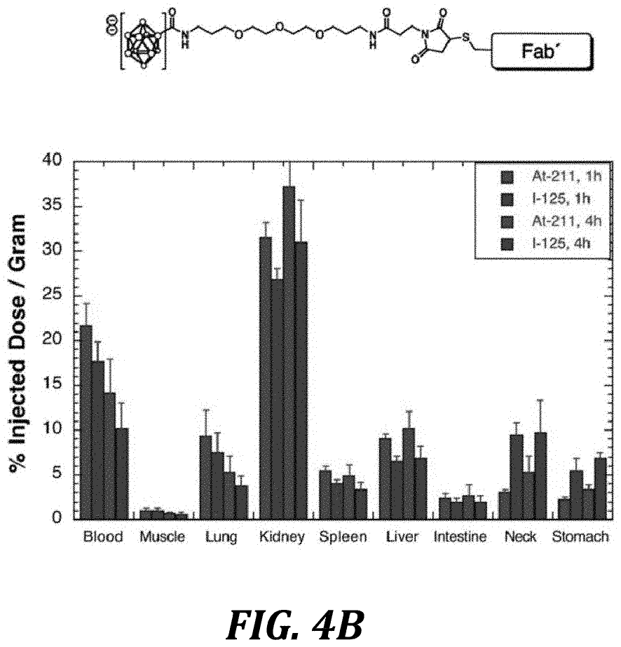

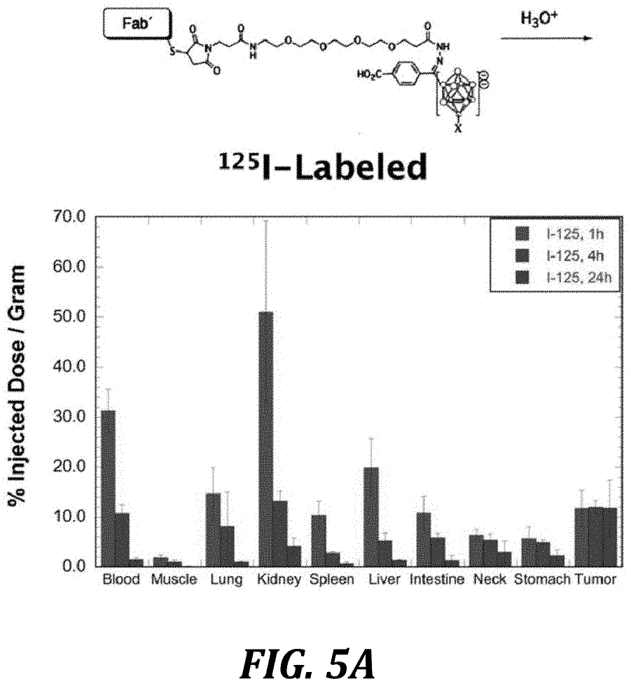

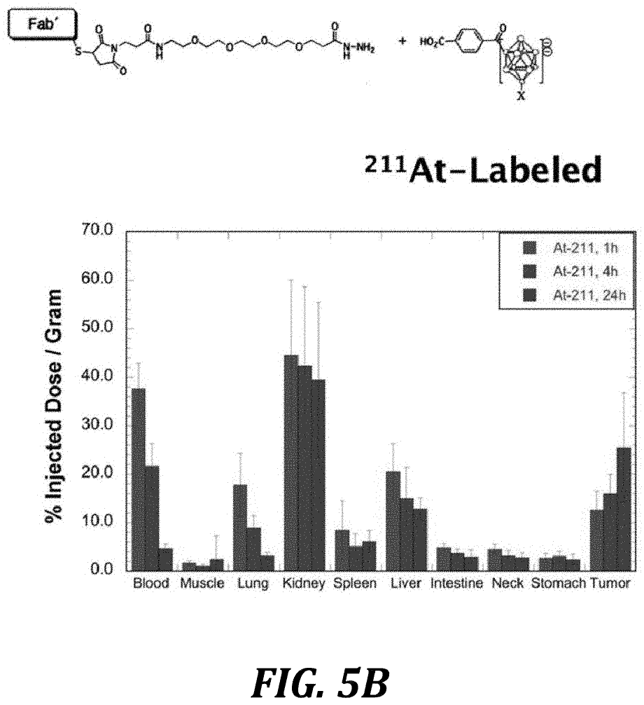

The rationale for pre-oxidizing molecular bound astatine atoms is not just predicated on formation of MAb-B10 dimers. Most importantly, it is also based on the observation that molecules containing astatine can remain in tissues much longer that the same molecule labeled with radioiodine. A particularly striking example of this was noted when evaluating an astatinated Fab′ antibody conjugate that had a closo-decaborate (2-) moiety attached on a cleavable linker. When a MAb Fab′ is labeled with both radioiodine and astatine on a non-cleavable linker, one generally obtains biodistributions such as shown in FIGS. 4A and 4B, where the kidneys have the highest concentration of both radioiodine and astatine. The high kidney concentrations led to research into methods to release the radioactivity from the kidneys. A particularly attractive approach to release of kidney-localized radioactivity was to prepare a Fab′ conjugate in which the closo-decaborate (2-) moiety was attached to an acid cleavable hydrazone linker. Initially several “cleavable” linkers containing the B10 moiety were evaluated, and a hydrazone-linked keto-benzoate derivative appeared to have the best in vivo characteristics. A thio-reactive maleimide linker was prepared containing the same hydrazone functionality and that linker was conjugated with a Fab′. The Fab′ conjugate was labeled (separately) with ¹²⁵I and ²¹¹At, and those radiolabeled Fab′ conjugates were co-injected into mice. Bar graphs showing the tissue concentrations of both radionuclides at 1, 4 and 24 h post injection are shown in FIGS. 5A and 5B. While the radioiodine cleared from all tissues, including kidney, the astatine did not clear from tissues in which the Fab′ was metabolized, including kidney and liver. This difference in radioactivity release from tissues is most likely due to oxidation of the astatine atom bound with the borate cage followed by reaction with cellular components to residualize the activity in the tissue.

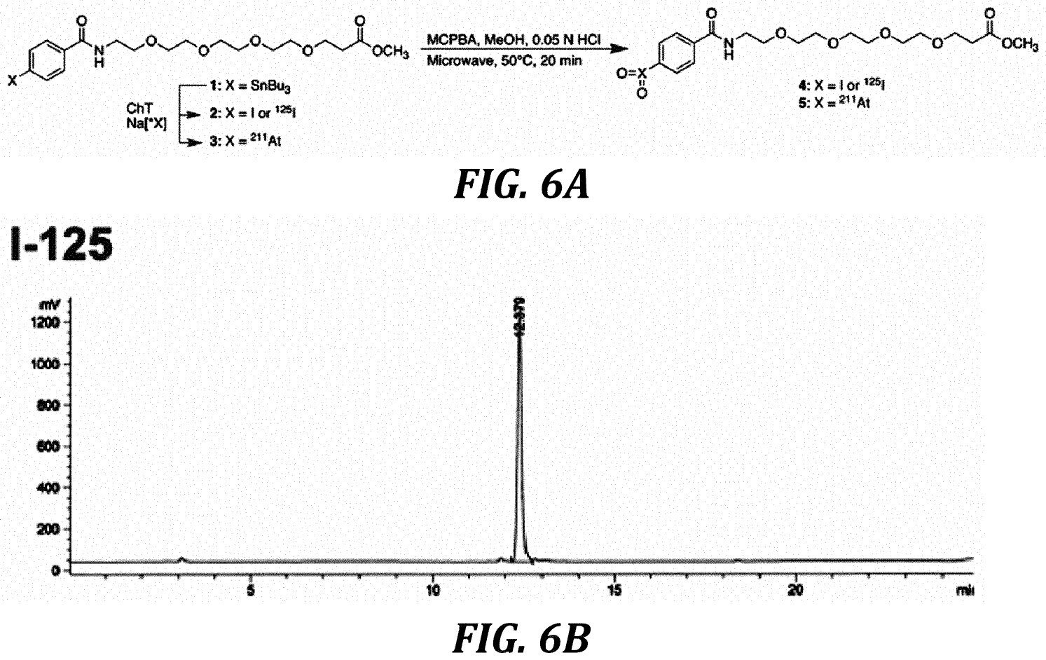

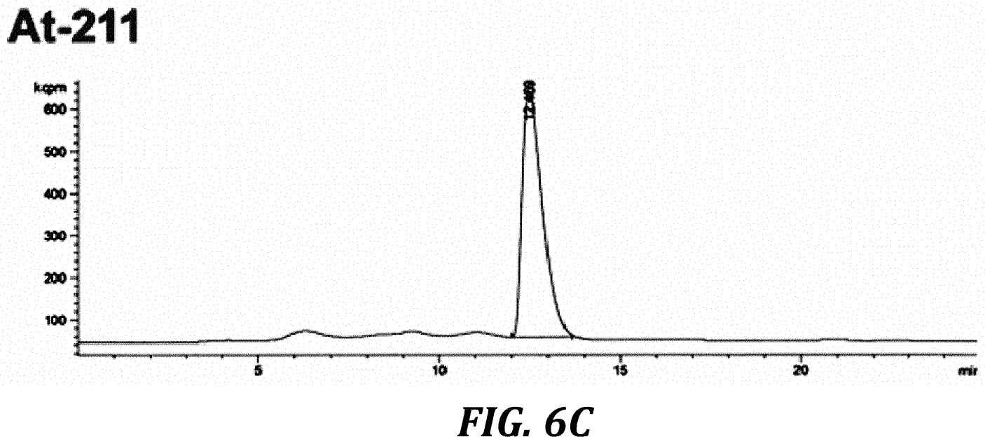

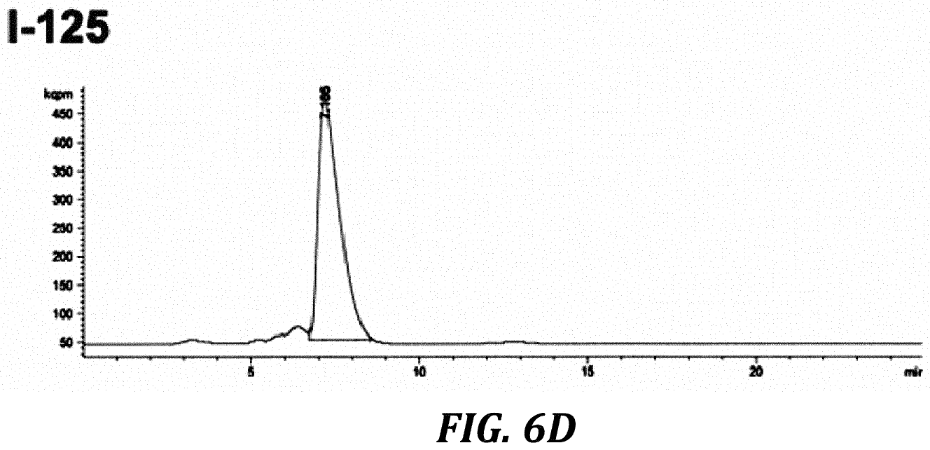

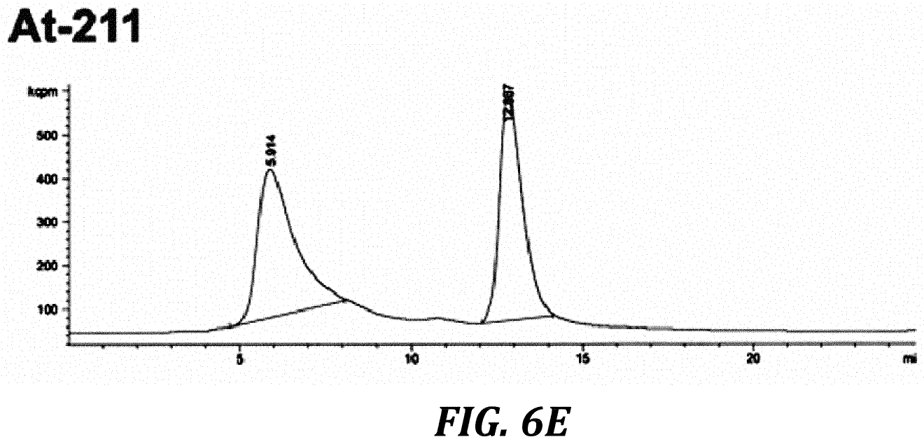

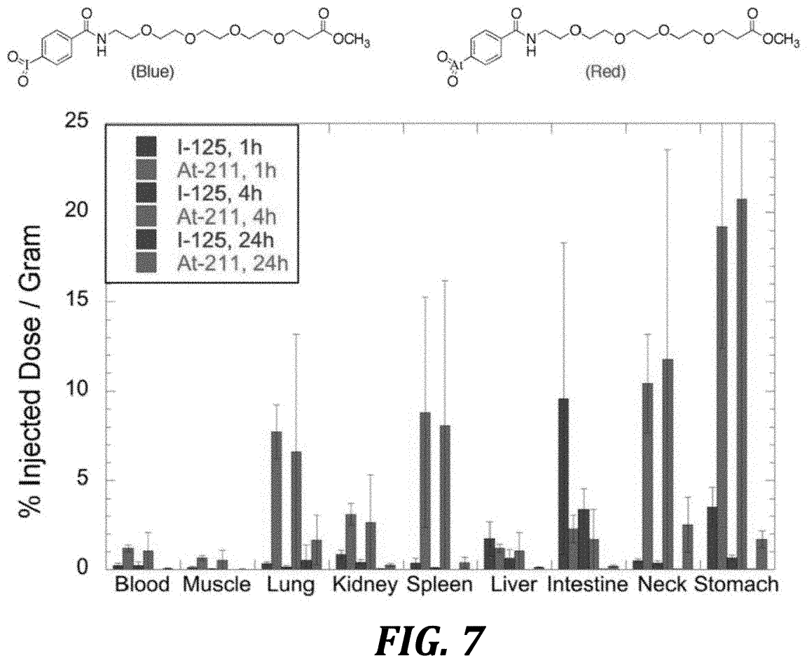

It is important to note that the type of the atoms bonded to the astatine atom in a higher oxidation state is critical. It was known that phenyliodoso compounds which have iodine in the +3 andiodoxybenzene in +5 formal oxidation state, could be made. In a study involving oxidation of (radio)iodine- and astatine-labeled electron-deficient compounds, new chemical entities were obtained as shown in reversed-phase chromatograms, as shown in FIGS. 6A-6E. The radioiodinated (¹²⁵I) and astatinated (²¹¹At) compounds had very similar retention times (e.g., 12.4 and 12.5 min, respectively), whereas after oxidation with meta-chloroperbenzoic acid (MCPB at 50° C. for 20 min), products with quite different retention times were obtained. Since a fully characterized reference compound was prepared using stable iodine (compound 4), the structure of the radioiodinated compound at t_{R}=7.2 minis known. In contrast, under the same oxidative reaction conditions obtaining an oxidized astatinated product was slower but provided a new product at 5.9 min retention time. The product may be the astatinated aryl compound, 5, or it may be astatate since astatate has a retention time similar to that of the new compound. Irrespective of which astatine compound it was, a biodistribution study was conducted after isolation of the early radio-HPLC peaks. Once the radioactive peaks were isolated the solvent was removed, then residues were taken up in phosphate buffer saline (PBS), mixed together and co-administered in groups of mice. The mice were sacrificed at 1, 4 and 24 hours post injection and selected tissues were obtained and counted at those timepoints. The results of the biodistribution study are shown as a bar graph in FIG. 7. It can be readily noted that the ¹²⁵I- and ²¹¹At-labeled products (radioactivity from HPLC) had very different tissue concentrations. The radioiodinated compound 4 (blue on graph) had low concentration in all tissues except in the intestines. In contrast, the astatinated compound (peak at 5.9 min) had high concentrations in lung, spleen, neck and stomach. It is important to note that high concentrations of astatine is also obtained in these tissues when free astatide is injected into mice. It would be expected that the corresponding radioiodinated compound in the +1 oxidation state (i.e., [¹²⁵I]2) would be stable to in vivo deiodination, and it appears that the iodinated aryliodoso compound in the +5 oxidation state (i.e., [¹²⁵I]4 is as well. The astatine-labeled molecule in the +1 oxidation state (i.e., [²¹¹At]3) would be expected to be unstable to in vivo deastatination, and it appears that the astatinated compound with a formal oxidation state of +5 (i.e., [²¹¹At]5) is also unstable in vivo or may actually be unstable to in vitro oxidation. These results indicate that oxidation of astatinated aryl compounds can make an astatinated compound unstable in vivo, or it may even release it from the compound prior to isolation of the activity.

The foregoing observations highlight the need to make the molecule-bound astatine resistant to oxidation. An avenue to achieve that is to “pre-oxidize” the astatine by building molecules that contain astatine to a higher oxidation state that is resistant to further oxidation. This disclosure describes the rationale and methods used to prepare astatine-labeled compounds in which the astatine is in a formal oxidation state of +3 or +5.

There are 34 known isotopes of astatine, but there are no stable isotopes and only two are considered appropriate for medical application at this time. The longest-lived isotope ²¹⁰At, has a half-life of 8.1 hours, but this isotope is not appropriate for human use as it decays to toxic ²¹⁰Po. The next longest-lived isotope ²¹¹At, with a half-life of is only 7.2 hours, has appropriate emissions for therapeutic use in patients. Another isotope of astatine, ²⁰⁹At which has a half-life of only 5.4 hours, has emissions that can be used for diagnostic imaging in patients. It is important to note that the two astatine isotopes must be man-made. The fact that there are no stable isotopes has made it particularly difficult to prepare and fully characterize chemical species of astatine and to develop methods for attaching it to disease targeting agents. This has been made even more difficult due to the fact that astatine isotopes must be made on special equipment. Unfortunately, the only method of identifying astatine chemical species or the nature of labeled compounds has been to compare chemical, chromatographic and biologic behavior of astatine compounds with similar iodine compounds. However, it turns out that iodine is often not a good surrogate for astatine, and results obtained can be difficult to interpret for this enigmatic element. In recent years mathematic modeling has provided information on what species might be present, but to date it has been very difficult to verify the predictions made through modeling. For instance, mathematical modeling has predicted that under acidic conditions the species present is AtO⁺ and not At⁺. This may be important as the product from electrophilic astatination reactions (e.g., R—At═O) might have a different chemical composition than has been reported (i.e., R—At) in the literature. It is very well established that electrophilic iodination (and radioiodination) results in R—I and not R—I═O, pointing out a potential difference between the elements that may support the contention that iodine is not always a good surrogate for astatine.

Although a great deal is known about iodine bonding to molecules in higher oxidation states, very little is known about astatine bonding in higher oxidation states. Iodine can exist in oxidation states of −1, 0, +1, +3, +5 and +7. Similarly, astatine can exist in −1, 0, +1, +3, +5 and +7. A change in astatine oxidation state that would be most favorable is oxidation from the formal +1 to formal +3 state, as that is the most readily achieved. This formal oxidation can be accomplished by reactions that result in bonding 2 or 3 atoms to a single astatine atom. It should be emphasized that, while astatine chemistry is similar to its nearest halogen neighbor, iodine, in many aspects, oxidation might be expected to be dissimilar due to the differences in outer electronic shells. Unfortunately, the differences are not well understood at this time. Therefore, the oxidation state obtained must be deduced by the nature of the reactants used and products obtained. Fortunately, the proximity of iodine and astatine in the periodic chart and the similarities of iodine chemistry to astatine's, supports the use of well characterized iodine species and iodinated compounds as chromatographic standards for astatinated compounds. However, similarities in chromatographic characteristics of astatine and iodinated compounds does not allow one to unequivocally say they are the same.

When considering what type of atom might best be bonded to the astatine atom, one can evaluate how different groups attached to an iodine atom in the higher oxidation states affect its nature. While iodine chemistry and astatine chemistry may differ, general trends are often followed. In an extensive review of the chemistry of polyvalent iodine, a large number of compounds are described where iodine is in the +3 oxidation state (known as λ³-iodanes) and a smaller number of iodine-containing compounds wherein the iodine atom is in the +5 oxidation state (λ⁵-iodanes). The iodanes are classified differently based on the types of “ligands” that are attached to the iodine atom, and their chemistry is defined by different stability and reactivity. For example, iodosylarenes and its acyclic derivative (compounds A and B in Table 1) are strong oxidizing agents, which makes them unstable for in vivo use. The heterocyclic iodanes C and D are more stable than A or B, but they are also oxidizing agents making them questionable for in in vivo use. Note that in compound D the iodine atom is in the +5 oxidation state. The most stable polycoordinate iodine species is found in aryliodonium salts, e.g., compound E.

TABLE 1
Examples of polyvalent iodine compounds that have +3
and +5 oxidation states of iodine.

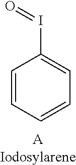

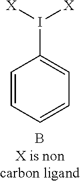

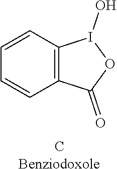 [SMILES: O=C1OI(O)c2ccccc21 ; "CBenziodoxole"]

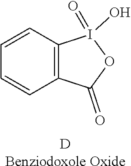 [SMILES: O=C1OI(=O)(O)c2ccccc21 ; "DBenziodoxole Oxide"]

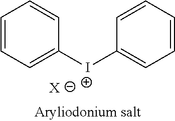

It can be noted that the examples of iodine compounds with higher formal oxidation states of iodine shown in Table 1 all have iodine attached to an aryl group. This is because aryl iodine provides higher stability in iodine compounds. This is also true for astatinated compounds, so the majority of astatinated compounds developed for in vivo use are aryl astatine compounds. The type of reaction used to prepare aryl astatinated compounds is important. Both nucleophilic and electrophilic substitution reactions of astatine provide labeled molecules. As electrophilic reactions with astatine are much more rapid and generally provide higher radiolabeling yields than nucleophilic reactions, this is the reaction type of choice for most astatinations. Radiolabeling of aromatic molecules with astatine requires that the molecule contain functional groups that activate it towards reaction with astatine or that an astatine-reactive pendant group be attached to the molecule that is to be astatinated. A large number of pendant groups have been developed to radiolabel aromatic compounds with astatine. However, in vivo stability has been a major obstacle for developing astatine-labeled agents for imaging and therapy of human diseases. This instability, when the astatine atom is bonded to a phenyl group, is believed to be brought about by oxidation of the astatine atom by cellular oxidase enzymes. A method for overcoming the in vivo oxidation is to pre-oxidize the molecule-bonded astatine atom to its formal +3 or +5 oxidation state. Similar to iodine, this can be accomplished by bonding 2, 3 or 4 atoms to the astatine atom.

The in vivo results obtained from oxidation of an astatinated aryl compound demonstrated that iodinated counterparts to astatinated compounds are not always good surrogates to use as predictors of what an astatinated compound might do in vivo. Perhaps more importantly, it points to a requirement to use different ligands to obtain stable astatinated compounds in a higher oxidation state. As with diaryliodonium salts, diarylastatonium salts can be predicted to be stable. This prediction had led to development of astatinated compounds of higher oxidation states that have two aromatic compounds bonded with a single positively charged astatine atom.

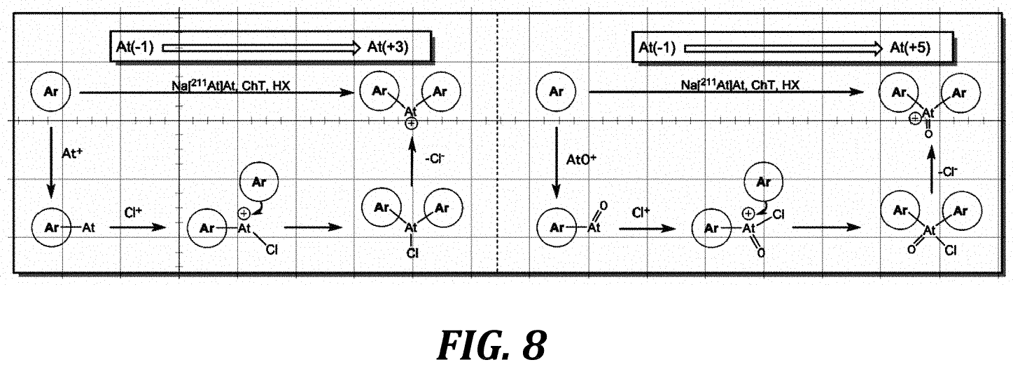

As discussed above for iodonium salts, compounds that have the diaryl astatonium “salt” structure can provide stable astatine-containing compounds for in vivo use. Generic schemes showing possible reaction pathways to provide diaryl astatonium salts are shown in FIG. 8.

An initial oxidation of astatide with an oxidant, such as chloramine-T (ChT), provides an electrophilic astatine species. Since the reactive electrophilic species in astatination reactions is not clear at this time, reactions with both At⁺ or AtO⁺ are shown in FIG. 8. In the first step, electrophilic reaction of At⁺ with an aryl compound (Ar) yields Ar-At and similarly reaction with AtO⁺ yields Ar—At═O. Since the electrophilic reaction replaces (or substitutes) an At atom for a proton (H+) on the aromatic ring, the resulting compound is neutral (not considering any other functional groups present). In the Ar-At molecule the astatine atom has a formal +1 oxidation state. In contrast, in the Ar—At═O molecule the astatine has a formal oxidation state of +3. A second oxidation step (e.g., Cl⁺) on the astatine atom in either of the aryl astatine compounds can result in the astatine atom becoming electropositive (formally in the +3 or +5 oxidation state, respectively). Electrophilic substitution of a second aryl compound or moiety can provide a tetra-substituted astatine atom, which can release a chloride atom to provide the diaryl astatonium salt in which the astatine atom has either a +3 or +5 formal oxidation state.

FIG. 8 shows reactions that can provide astatine diaryl products in either a +3 or +5 formal oxidation state based on whether At⁺ or AtO⁺ is the electrophilic species formed under the reaction conditions. These generic reaction sequences can be applied irrespective of the type of electrophilic aromatic rings being reacted. Additionally, depending on the charge on the aromatic group, the At in the final product may release the chloride ion to take on a positive charge, with a counterion of either Cl— or X—.

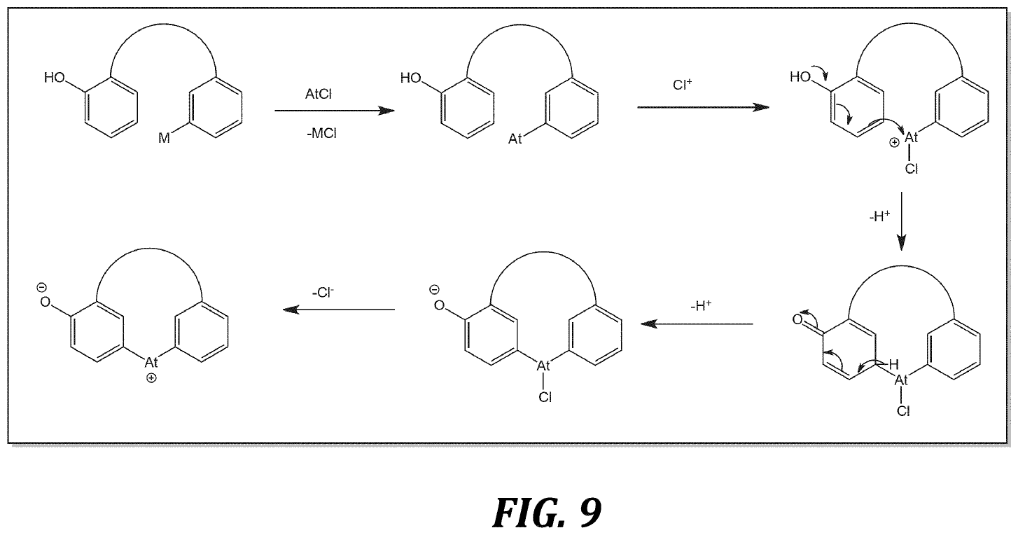

While two different compounds that contain aryl groups can be used to form the diaryl astatonium salt compounds, it is preferred that the two aryl moieties be part of the same compound. Having two aryl moieties in the same compound can facilitate the reaction as it is an intramolecular reaction and it assures that mixtures of products are not obtained. An example of a reaction that provides a diaryl astatonium salt that contains two aryl groups might be prepared is shown in FIG. 9. Note that the fact that the two aryl moieties are in the same compound is depicted by the arched line connecting them. In this example one aryl is electron deficient (non-activated) so an organometallic intermediate (M) is used to obtain the electrophilic reaction. The other aryl group is electron rich (highly activated phenol) allowing the electrophilic second step to occur spontaneously. In this example the organometallic intermediate has to be of high reactivity (e.g., R₃Sn or B(OH)₂) such that it reacts preferentially with it, rather than the phenol. If a lower reactive organometallic intermediate (e.g., SiMe₃) is used in the reaction, functionalization of the phenol such that it is deactivated (e.g. formation of an acetyl ester) would be used, then be removed post the initial astatination reaction. An ortho-phenol can also be used in this reaction, as well as other activated aryl moieties (e.g., anisole, aniline). If the molecule being astatinated does not have an activated aryl ring, two aryl organometallic intermediates can be prepared to allow cross-linking of the two aryl compounds with an astatine atom in a higher oxidation state.

Bis-aryl At (+3)-bridged compounds, such as those shown in FIG. 9, are stabilized relative to a single At-aryl bonding structure, but it is preferable that at least one of the aromatic ligands bonded to the astatine be a boron cage moiety to add a higher stability to the At bond. This design is based on the high in vivo stability of observed for compounds where astatine is bonded to a boron cage moiety. It is also important that the aromatic boron cage moiety have a negative charge as found in nido-carboranes, monocarbon carboranes, or borates. This requirement is brought about by the fact that aromatic boron compounds that have no charge have extremely low aqueous solubility and have very low reactivity in electrophilic reactions. Anionic boron cage moieties, due to the charge on the aromatic structure, generally undergo electrophilic substitution reactions. Indeed, nido-carboranes (1-) and closo-decaborate (2-) moieties react so rapidly that compounds containing them can be conjugated with intact monoclonal antibodies for direct astatine labeling even in the presence of a large number of tyrosine (phenolic) residues. Observations when labeling monoclonal antibodies conjugated with closo-decaborate (2-) moieties have further supported the use of astatine labeling in higher oxidation states.

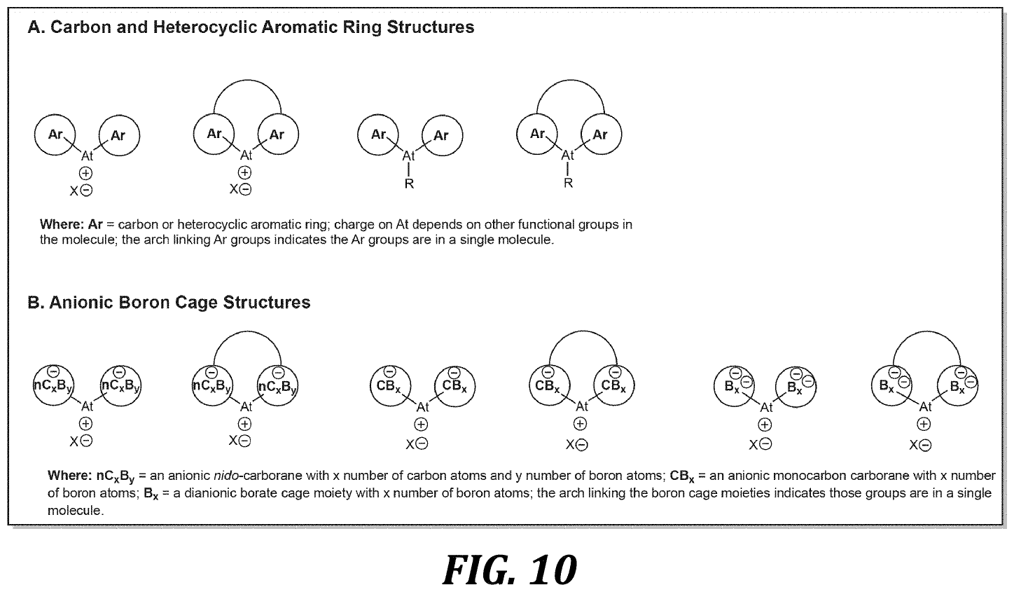

There are a number of different combinations of aromatic ring compounds, that when astatinated, can provide astatine (+3) or astatine (+5) labeling. FIG. 10 has generic depictions showing astatinated compounds wherein astatine is bonded to two aromatic ring compounds or heteroaromatic ring compounds (Ar), two nido-carboranes (nCₓB_{y}), two monocarbon carboranes (CBₓ), or two closo-borates (Bx). Other combinations of these astatine-reactive aromatic moieties are also possible, e.g., Ar—At—CBₓ, CBₓ—At—Bₓ, etc. While all of these structures can provide stable astatine (3+) molecules, the examples wherein the aromatic moieties are in a single molecule are preferred as that provides the best configuration to attain the desired reaction with the activated astatine atom.

Compounds in nature do not contain aromatic anionic boron cage moieties, so they must be incorporated into pendant groups for conjugation to disease-targeting agents. Since the preferred structure for these pendant groups is to have both of the aromatic ring structures in the same molecule, pendant groups must contain both of the aromatic moieties.

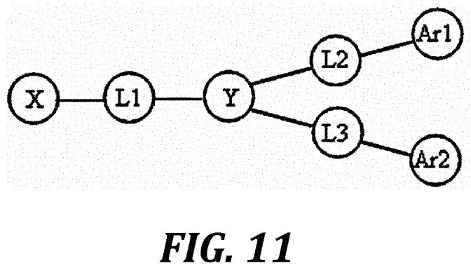

A generic representation of the composition of a pendant group is shown in FIG. 11, where:

X=a functionality reactive with a functional group on a disease-targeting agent, such as an active ester, phenyl isothiocyanate, maleimide;

L1=a linking group that is bonded to X and Y, this may be used to provide a distance between X and Y, it may have functional groups within it to impart aqueous solubility and/or ionic charges Y=a trifunctional group of atoms or trifunctional aryl moiety that is bonded to the linking groups L1, L2 and L3;

L2=a linking group that is bonded to Y and Ar1, this may be used to provide a distance between Y and Ar1, it may have functional groups within it to impart aqueous solubility and/or ionic charges;

L3=a linking group that is bonded to Y and Ar2, this may be used to provide a distance between Y and Ar2, it may have functional groups within it to impart aqueous solubility and/or ionic charges;

Ar1=aromatic moiety, anionic aromatic moiety or modified aromatic moiety that is reactive with electropositive astatine, this includes aromatic moieties such as phenol, thiophenol, aniline, anisole, organometallic substituted benzene, nido-carborane, monocarbon carborane, closo-borate (2-); and

Ar2=aromatic moiety, anionic aromatic moiety or modified aromatic moiety that is reactive with electropositive astatine, this includes aromatic moieties such as phenol, thiophenol, aniline, anisole, organometallic substituted benzene, nido-carborane, monocarbon carborane, closo-borate(2-).

### The Preparation and Characteristics of Representative Astatine-Containing Conjugates: Use of Bis-B10 Conjugate of MAb to Stop Dimerization of MAb

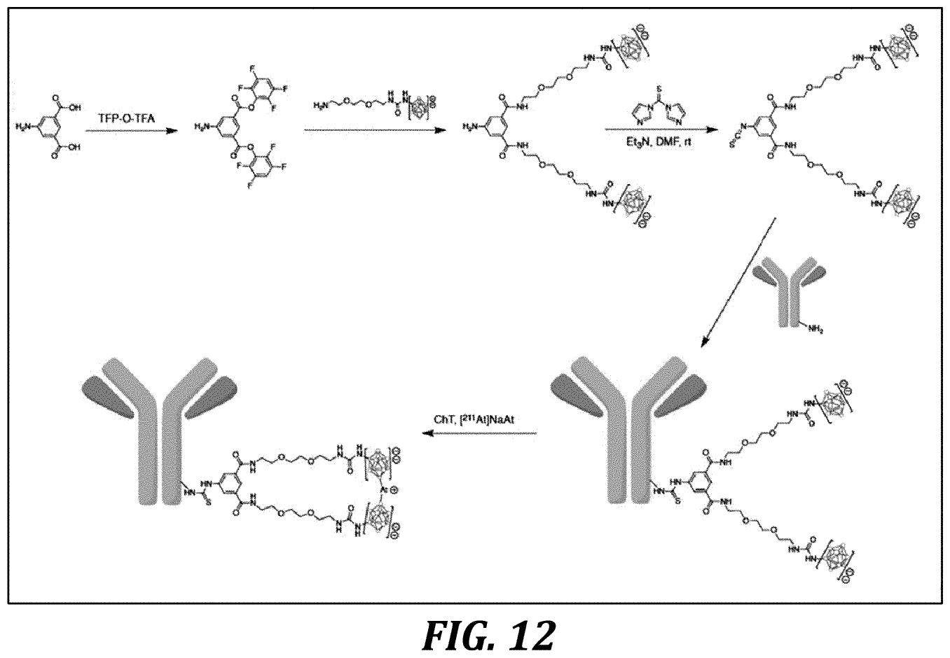

In FIGS. 2A-2D the effect of using an oxidant (chloramine-T) when conducting astatination reactions on antibodies conjugated with closo-decaborate (2-) moieties was exemplified through radiochromatograms, which show the quantity of dimer formed in astatination reactions with and without oxidant. To alleviate the issue of cross-linking antibodies that contain closo-decaborate (2-) conjugates, it was determined that providing two closo-decaborate(2-) moieties on the same conjugate would work. Thus, an isothiocyanato-phenyl conjugate that contained two closo-decaborate (2-) moieties was synthesized, conjugated with an antibody, and astatinated as shown in FIG. 12.

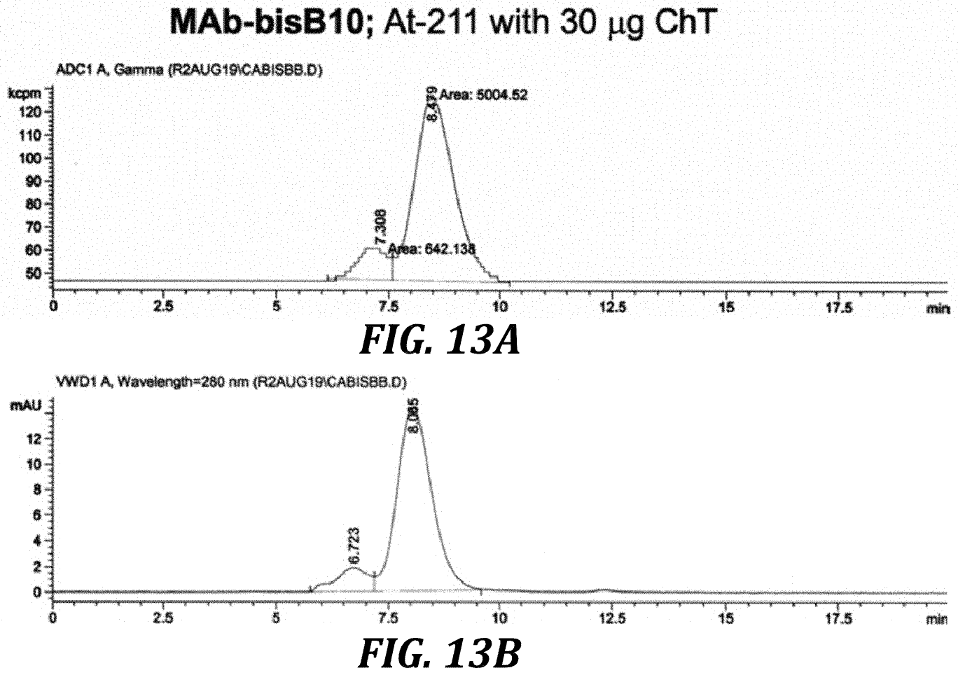

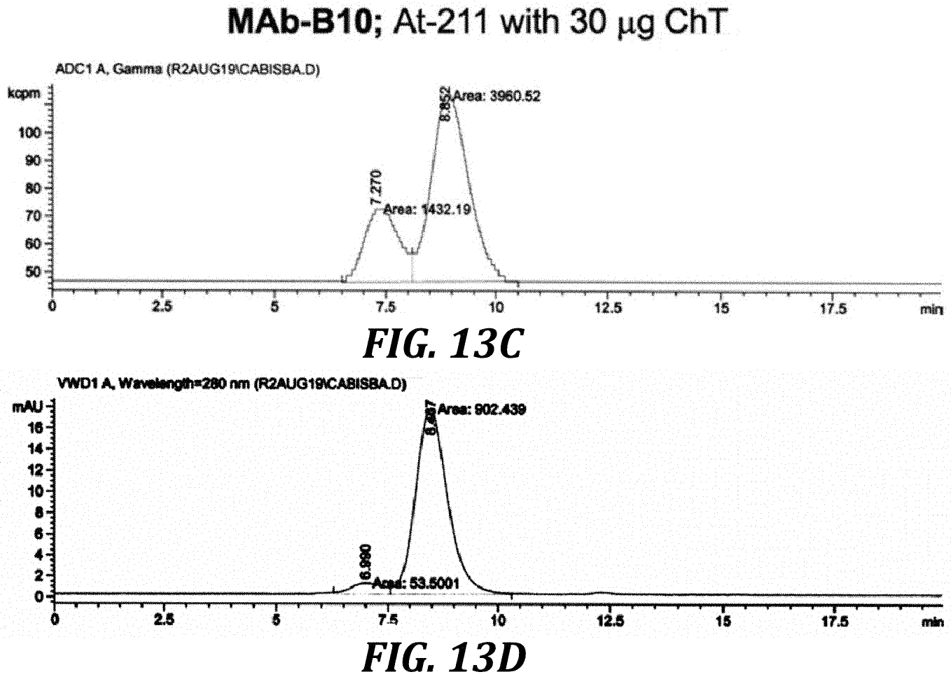

The use of a bis-aryl antibody conjugate can prevent cross-linking (dimerizations as shown in FIGS. 2A-2D) when an oxidant such as chloramine-T (ChT) is used in the astatination reaction was tested in the following experiment. A 500 ug sample of CA12.10C12 conjugate (either CA12.10C12-B10 or CA12.10C12-bisB10) in PBS was added to a solution containing 100 uL of sodium phosphate, pH 6.8, and 100 uL of a neutral aqueous [²¹¹At]NaAt solution (1 mCi), then 30 ug of ChT (30 uL of a 1 mg/mL aqueous solution) was added. The reaction was allowed to go for 5 min, then 30 uL of a 1 mg/mL solution of sodium metabisulfite was added to quench. The ²¹¹At-labeled MAb was purified on a PD-10 eluting with PBS. The purity of the isolated ²¹¹At-labeled CA12.10C12-B10 and CA12.10C12-bisB10 was assessed by size-exclusion radio-HPLC. The radiochromatograms are shown in FIGS. 13A and 13C. The results clearly show that there is no increase (over UV %) of a radiolabeled higher molecular weight species when CA12.10C12-bisB10 was labeled with ²¹¹At using ChT, whereas there was a −21% increase in higher molecular weight species when the monoB10 conjugate CA12.10C12 was astatinated under identical reaction conditions. These results support that an astatonium salt is formed as shown in FIG. 12. This an example where astatine is converted to the +3 oxidation state to decrease the side reaction products obtained.

The example of using the astatonium salt formation to decrease side reactions when labeling monoclonal antibody conjugates is not where it will be most advantageous, as one can label the antibody conjugate with ²¹¹At without added oxidant and separate the dimer from it. The most important aspect to using a higher oxidation state of ²¹¹At is that it will not be further oxidized in vivo so ²¹¹At-labeled compounds will not be trapped in tissues, resulting in them behaving more like their radioiodinated surrogates. In that case, the in vivo pharmacokinetics and tissue distribution of an ²¹¹At-hydrazone conjugate of a Fab′ fragment would look like the ¹²⁵I-labeled compound (FIG. 5A) and not like that of the mono-B10 conjugate (FIG. 5B).

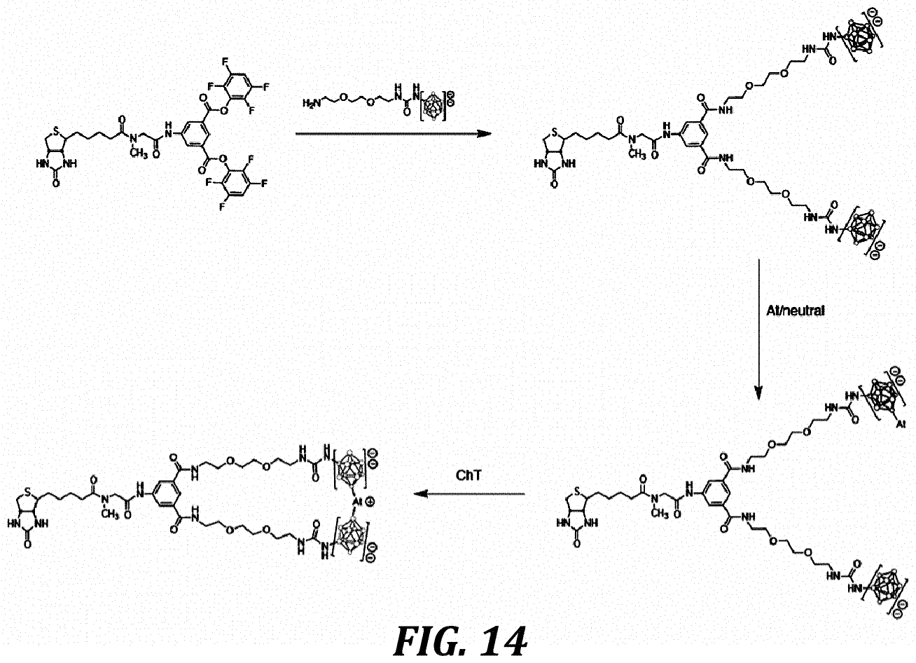

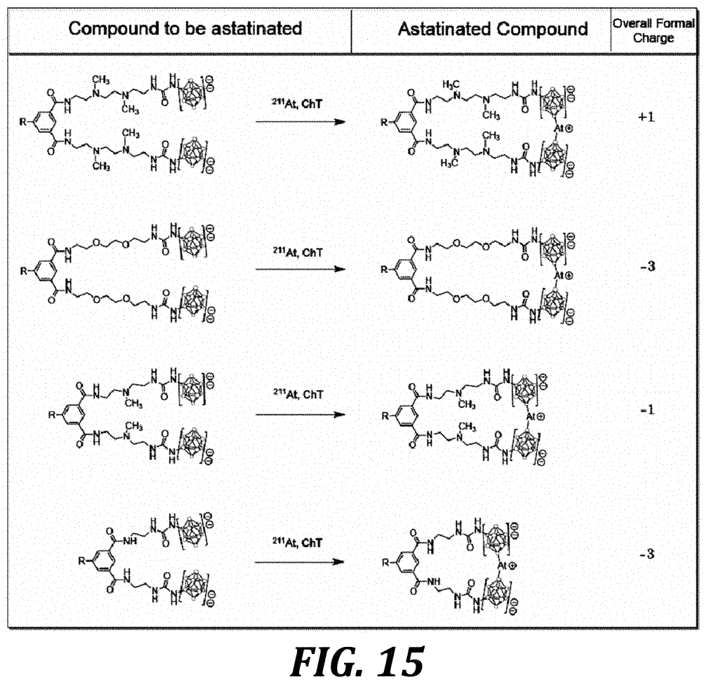

Astatinated biotin derivatives are of high interest for an approach to cancer therapy termed “pretargeting”. This is an example where having an astatonium salt attached to two boron atoms would be of great value. An example of such a derivative is shown in FIG. 14. The highly negative charge on the biotin derivative, and other compounds containing the aryl-bisB10 structure which has the aromatic ring linked to the B10 moiety through PEG moieties, will impart unfavorable characteristics to the compound. Therefore, alternate linking moieties, such as those in FIG. 15 can be used to change the overall charge on the aryl-bisB10 portion of the molecule. The composition of the linking arms can also change the lipophilicity of this portion of the molecule. For example, a large difference in lipophilicity can be expected in compounds that contain the diamino-dPEG2 linker vs. a diamino-ethane linker.

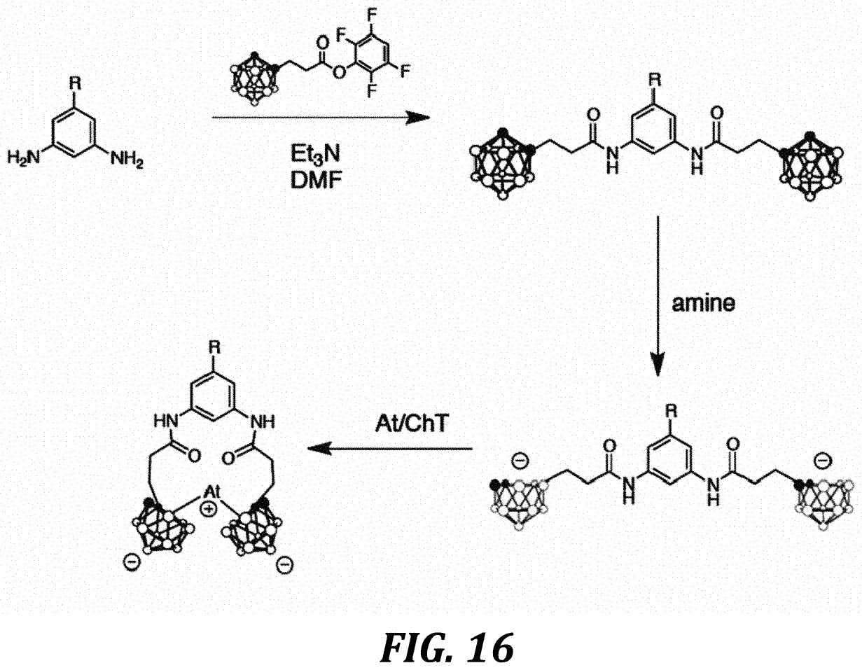

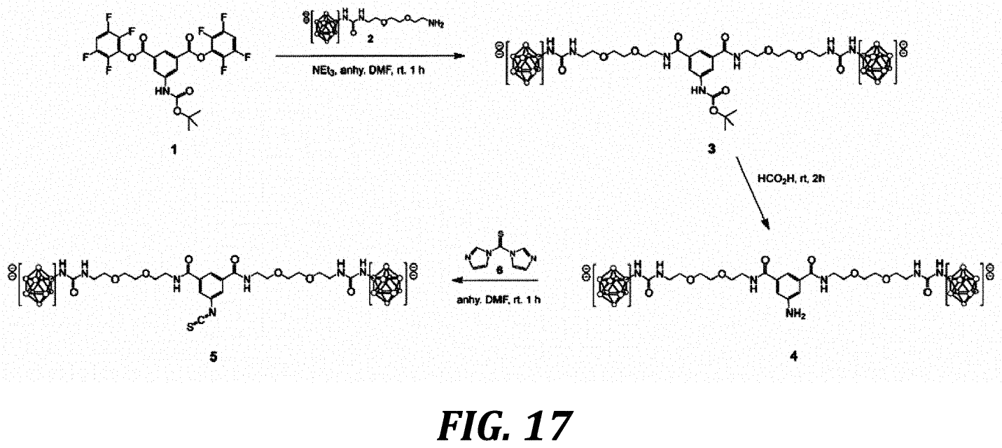

Other boron cage moieties can be used to prepare bridged astatonium salts for stability and non-reactivity in cells. An example of that is anionic nido-carboranes as shown in FIG. 16. In this case the mono-anionic nido-carborane can be prepared before or after synthesis of a bis-carborane by removal of a boron atom. Previous studies have shown that a bis-nido carborane (Venus flytrap) was stable in vivo, but it was highly lipophilic so other derivatives need to be prepare. As with the bis-B10 derivatives, linker molecules can impart more favorable lipophilicity.

The foregoing examples are only a few of the many possible examples of astatine-labeled molecules having two aromatic boron cage molecules attached to linker moieties through a trifunctional molecule can be envisioned.

Evaluation of Methods to Decrease Formation of a Higher Molecular Weight Species when ²¹¹At-Labeling of Antibody-810 Conjugates Using Chloramine-T as Oxidant

A boron cage labeling moiety, the isothiocyanato-phenethyl-ureido-closo-decaborate (2-) (abbreviated as B10-NCS) can be conjugated with MAbs, and ²¹¹At-labeled using chloramine-T (ChT), to provide ²¹¹At-MAb-B10 conjugates with high in vivo stability of the ²¹¹At label. However, in the studies it was observed that a radiolabeled high molecular weight (HMW) species was formed based on SE-HPLC chromatograms. Of note is the fact that the percent of the ²¹¹At-labeled HMW species in the radiochromatogram was considerably higher than the percentage of HMW species on the UV (280 nm) chromatogram. This fact suggested that the HMW species formation was unique to ²¹¹At. Indeed, the reaction of the same MAb-B10 with radioiodine under the essentially identical reaction conditions (using ChT) did not show a difference between the HMW percentage in the radiochromatogram and the UV chromatogram. The quantity of ChT oxidant used may be problematic, so different quantities of ChT in the labeling reaction were evaluated. A new conjugation moiety that contains two closo-decaborate (2-) moieties was synthesized, conjugated to a MAb and radiolabeled with ²¹¹At to determine if it could be used to alleviate the ²¹¹At-labeled HMW species.

The B10-NCS reagent was prepared as previously published. The bis(B10)-NCS reagent was prepared in a multi-step reaction sequence. Two anti-CD45 MAbs were used in the studies, CA12.10C12 and BC8 MAb conjugations of the two different B10 reagents were conducted in PBS, pH 8.5 at room temperature overnight. The MAb conjugates were characterized by SE-HPLC and IEF. The MAb-B10 and MAb-bis(B10) conjugates were labeled with ²¹¹At, adding 20, 10, 5 or 0 μg of ChT to the reaction solution. SE-HPLC with UV and radioactive detection was used to evaluate products obtained in ²¹¹At-labeling reactions. ²¹¹At-labeling of MAb-bis(B10) was conducted with 20 μg of ChT.

²¹¹At-labeling of a MAb-B10 conjugate, CA12.10C12-B10, using decreasing quantities of the oxidant ChT (20, 10, 5, or 0 μg) resulted in high overall labeling radiochemical yields (90+%), with varying quantities (27%, 17%, 13%, and 9%, respectively) of a HMW species being formed. The high ²¹¹At-labeling yield when using no oxidant was confirmed in several MAb-B10 labeling reactions. Importantly, radioiodination of the same reagent using 20 μg of ChT had essentially no HMW species formed. Under the same reaction conditions, a MAb-bis(B10) conjugate had no HMW species formed when 20 μg of ChT was used in the ²¹¹At-labeling reaction.

Studies demonstrated that no oxidant is required to label MAb-B10 conjugates with ²¹¹At and high radiochemical yields are obtained in the reactions. The studies clearly showed that a lack of oxidant (ChT) in the ²¹¹At-labeling of MAb-B10 results in minimal or no formation of ²¹¹At-labeled HMW species. That result is further confirmed by the fact that the HMW species is not obtained when the MAb was conjugated with a reagent that contained two B10 moieties, MAb-bis(B10). The study results suggest that ²¹¹At-labeled HMW species seen by SE-HPLC may be a MAb-B10-At-B10-MAb dimer, where ²¹¹At is presumably forming an astatonium ion.

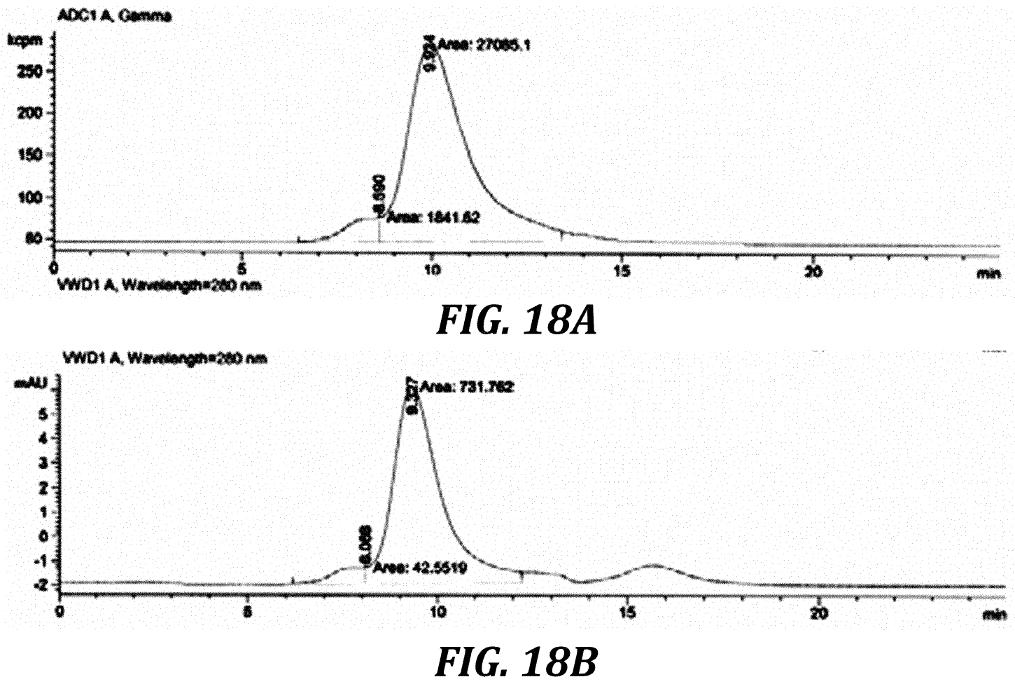

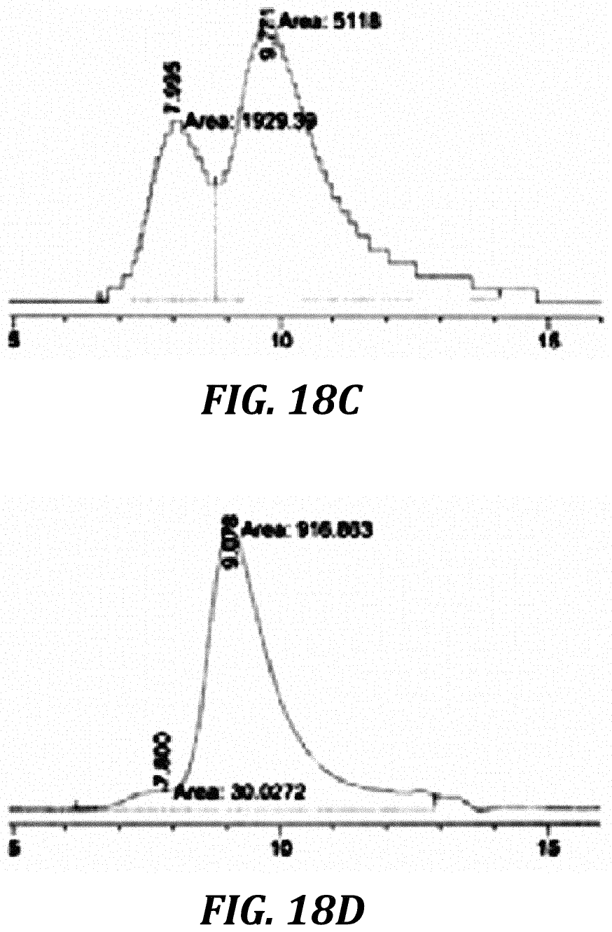

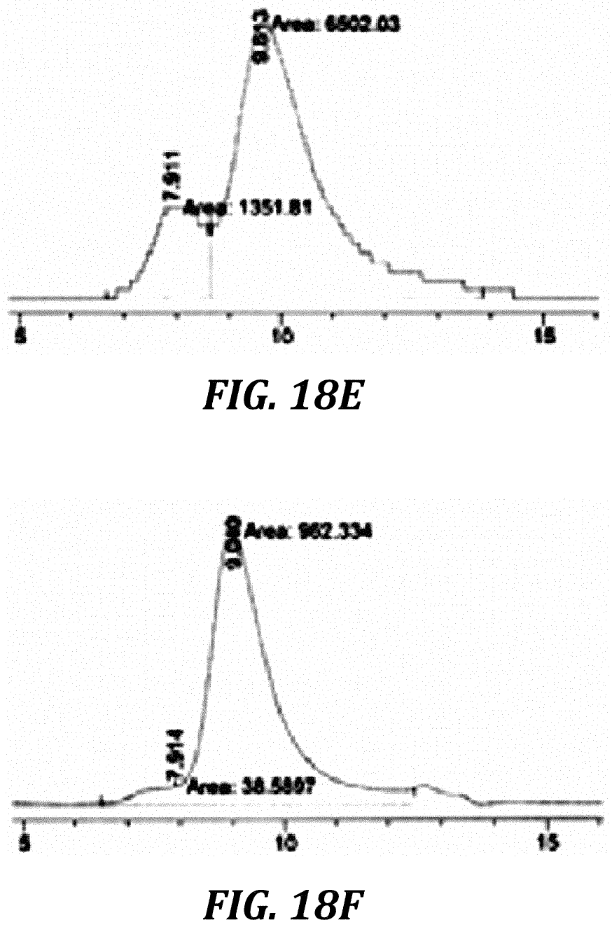

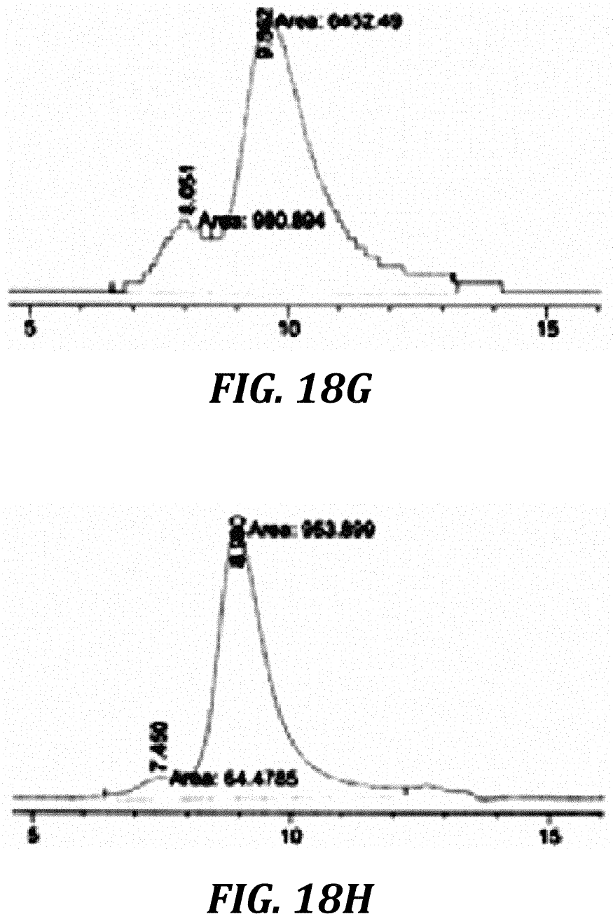

FIGS. 18A-18H compare size-exclusion HPLC chromatograms showing products from ²¹¹At- and ¹²⁵I-labeling of CA12.10C12-B10 (MAb-B10) using varying quantities of chloramine-T: FIGS. 18A and 18B, 125I labeling with 20 μg ChT; and FIGS. 18C-18H, ²¹¹At-labeling with 20 μg ChT (18C, 18D), 10 μg ChT (18E, 18F), and 5 μg ChT (18G, 18H). FIGS. 18A, 18C, 18E, and 18G are radiochromatograms and FIGS. 18B, 18D, 18F, and 18H are chromatograms using UV detection (280 nm).

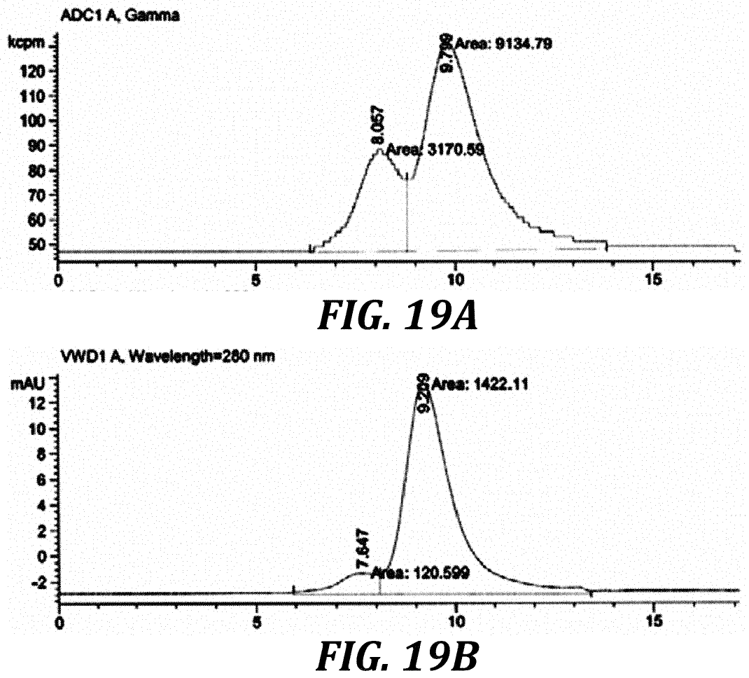

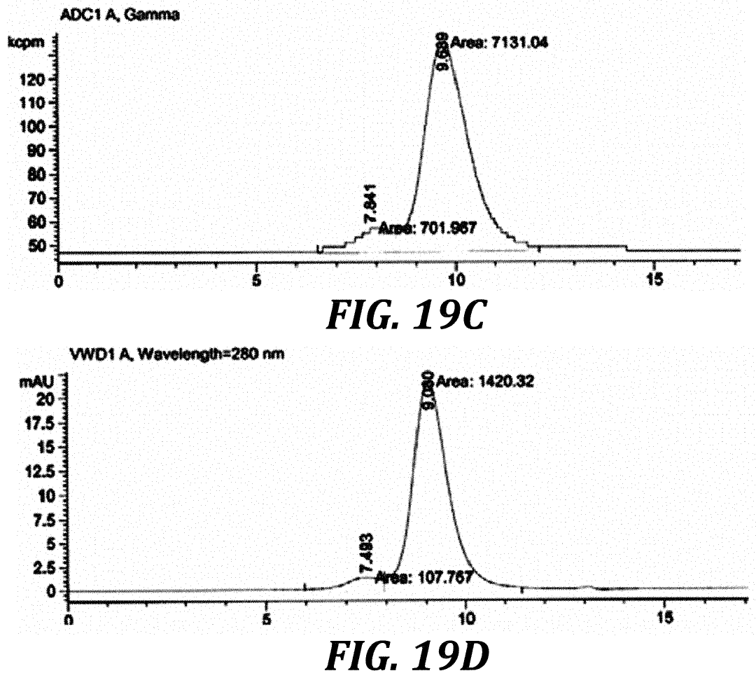

FIGS. 19A-19D compare size-exclusion HPLC chromatograms showing products from ²¹¹At-labeling of 8C8-B10 using 20 μg ChT (19A, 19B) or no ChT (19C, 19D). FIGS. 19A and 19C are radiochromatograms and FIGS. 19B and 19D are chromatograms using UV detection (280 nm).

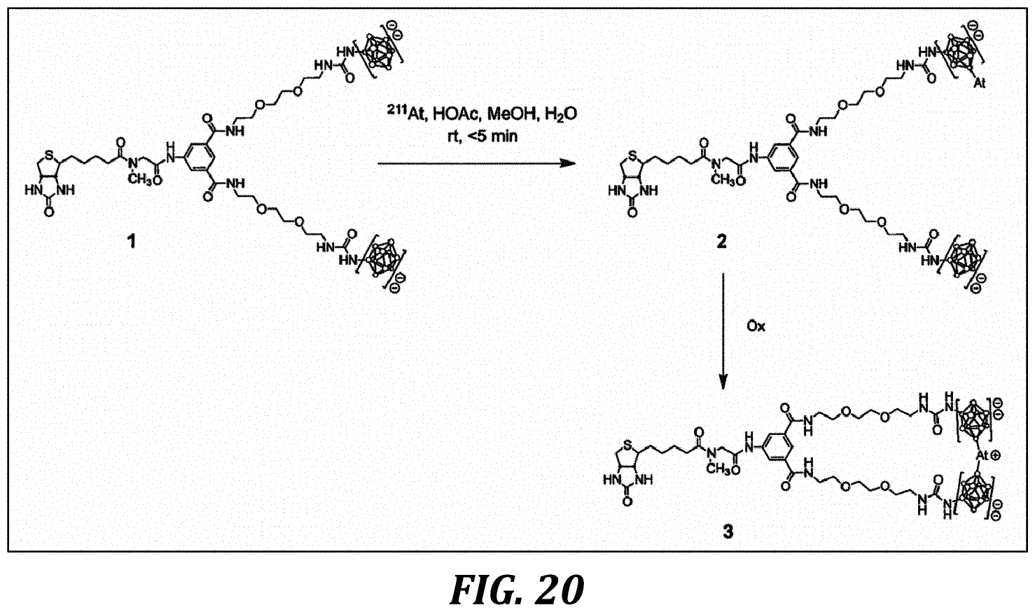

The in vivo stability of ²¹¹At-labeled compounds has been an impediment to developing new radiopharmaceuticals containing this γ-emitting radionuclide. Early studies demonstrated that labeling monoclonal antibodies using non-activated meta- or para-benzoyl or benzyl conjugates can provide adequate stability for use in vivo. However, when used on more rapidly metabolized molecules, such as antibody fragments, peptides or small molecules, high in vivo instability is seen for most molecules studied. To circumvent the instability, ²¹¹At-labeled aromatic boron-containing compounds were developed which provide excellent in vivo stability to loss of the astatine atom. One aromatic boron-containing moiety that has worked particularly well for in vivo application is the closo-decaborate (2-) moiety (referred to herein as B10(2-)). Despite this, large in vivo differences in tissue concentrations have been noted for some molecules conjugated to B10(2-) when labeled with radioiodine and separately with ²¹¹At. The in vivo oxidation of the astatine bonded to the B10(2-) moiety causes it to become reactive with cellular components and is retained in cells, whereas radioiodine bonded to the boron cage is not oxidized and not retained in cells. Referring to FIG. 20, biotin derivative, 1, that has two B10(2-) moieties was prepared having an astatonium ion between the two B10(2-) moieties. The astatine atom bonded to two B10(2-) moieties would be in the +3 oxidation state vs. the +1 oxidation state for single bonded astatine. Reaction of 1 with ²¹¹At in 5% HOAc/MeOH/H2O without an added oxidant proceeds in a stepwise manner by radio-HPLC analysis, presumably undergoing very rapid reaction with one B10(2-) moiety to form an astatinated compound 2, then reacting more slowly (30 min) with the second B10(2-) moiety to form the bis-B10 astatonium ion compound 3. See FIG. 20.

Bis-B10 Synthesis and ²¹¹At Labeling

As described herein, the formation of a bis-aryl astatonium ion in an ²¹¹At-labeled molecule can avoid in vivo oxidation that causes release of the ²¹¹At from the labeled molecule and/or reaction with cellular components that causes the ²¹¹At to be retained in the cell. This retention in cells is particularly problematic if the cells are in non-target tissues such as liver, kidney and lung.

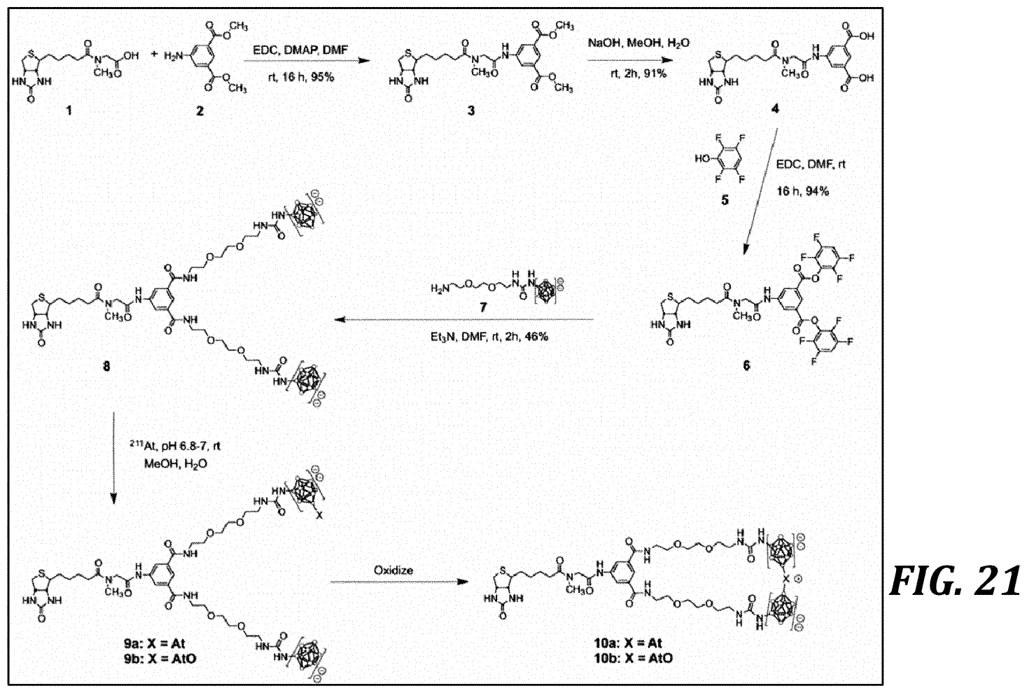

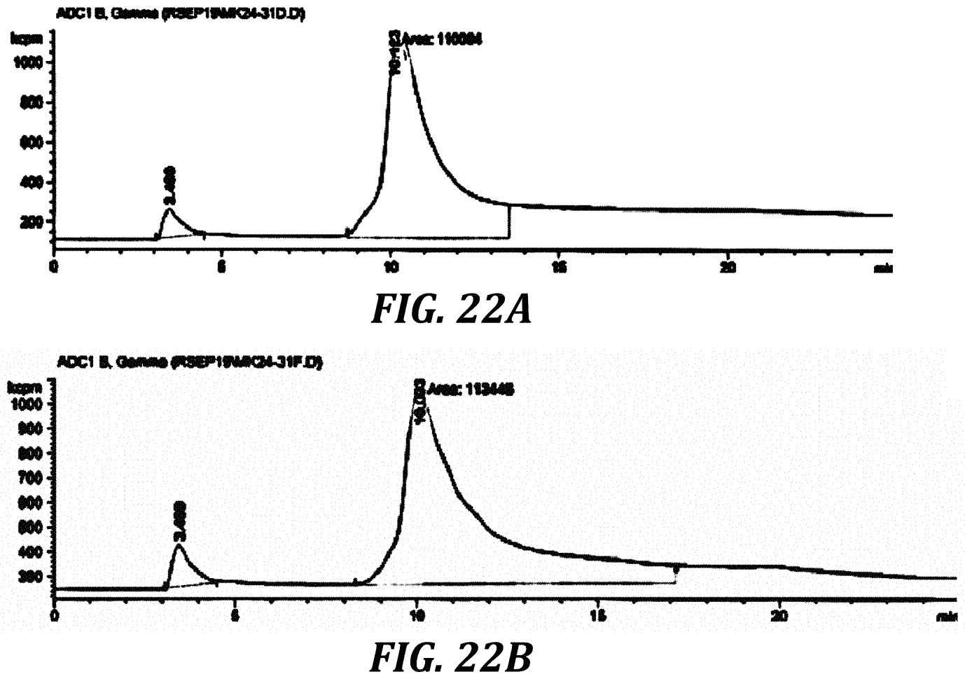

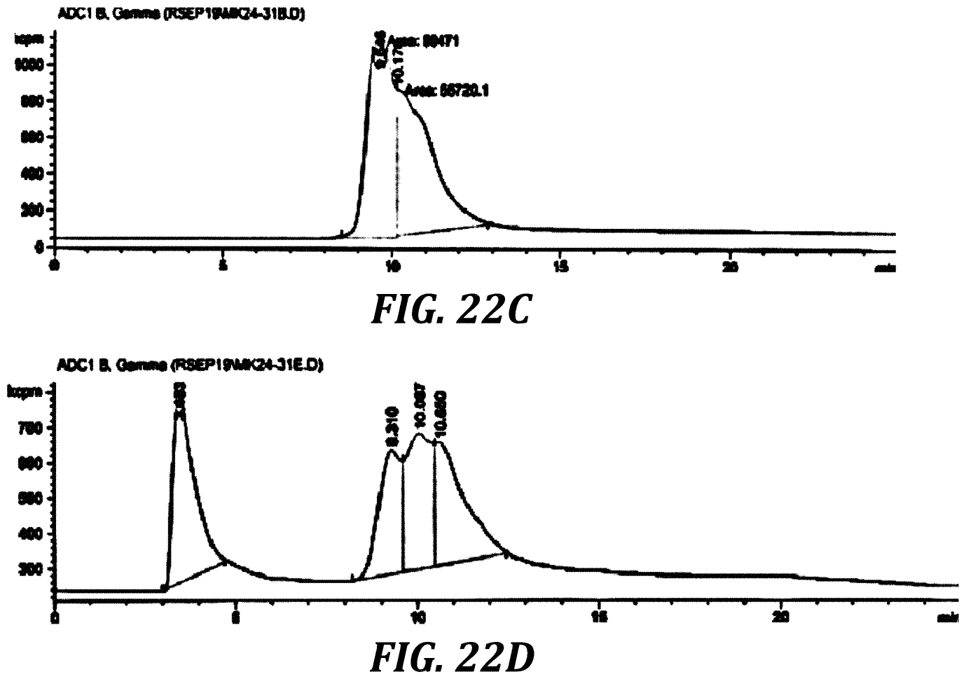

A biotin-sarcosine-bis-B10 adduct, 8, was synthesized as shown in FIG. 21 and described below. An initial ²¹¹At-labeled reaction with biotin derivative, 8, resulted in one primary labeled species being made (see FIG. 22A). In that product, 9, the ²¹¹At atom is either in the +1 oxidation state (9a, reactant At+) or in the +3 oxidation state (9b, reactant is AtO+) depending on the actual electrophilic agent in solution. Additional oxidation of the B10-bonded At atom can convert it to an electropositive species (stable when on a dianionic decaborate). The electropositive B10-At-X species in either 9a or 9b can undergo an intramolecular reaction with the second B10 moiety to form a bridged astatonium derivative (10a or 10b, structure will depend on whether the initial product is 9a or 9b). Ring closure from 9a provides 10a, where the At atom is in a +3 oxidation state. Ring closure from 9b to provide 10b, will result in the At atom being in a +5 oxidation state.

In an ²¹¹At-labeling reaction with biotin-sarcosine-bis-B10, 8, a solution of [²¹¹At]NaAt in 5% HOAc in MeOH/H₂O was reacted at room temperature for 5 min without an oxidant. The reaction was quenched with 10 μL of 1 mg/mL Na₂S₂O₅ (a reductant). The reaction provided one major ²¹¹At labeled product (95%) as shown the radio-HPLC peak at about 10.1 min (FIG. 22A). No change was observed in the product after 1 h in solution at room temperature (FIG. 22B). In another ²¹¹At-labeling reaction, the same reaction conditions as above were used except a chemical oxidant chloramine-T (ChT) was added to the reaction solution. The radio-HPLC chromatogram from this reaction is shown in FIG. 22C. The reaction with ChT provided multiple products that were eluted from 9-11 min. After sitting at room temperature for 2 h, a significant amount of free ²¹¹At was observed (29%) (FIG. 22D).

The ²¹¹At-labeling experiments conducted with biotin-sarcosine-bis-B10, 8, show that only one primary labeled product (R_{T} about 10.1 min) is obtained (by radio-HPLC) when the oxidant ChT is absent in the reaction mixture (FIGS. 22A and 22B). However, when ChT is added to the reaction mixture, two additional labeled species are observed on the radio-HPLC chromatograms. It is likely that the additional ²¹¹At-labeled species (9.3 and 10.0 min retention time) are due to the oxidation of the biotin moiety and/or further oxidation of the boron bonded At atom by ChT.

Another ²¹¹At-labeling reaction of biotin-sarcosine-bis-B10, 8, using the reaction conditions described above (without ChT) was conducted, but without addition of the quenching agent at 5 min. A sample from the reaction mixture after 5 min at room temperature displayed two radiolabeled products on the radio-HPLC chromatogram eluting at about 9.0 min and 10.4 min. A radio-HPLC chromatogram of a sample taken at 100 min reaction time showed that the radio-peak eluted around 9.0 min decreased from 38.7% to 4.5%, and another sample taken at 130 min reaction time showed that only one major product was present, at about 10.7 min retention time. The radio-peak at 10.7 min was isolated to provide a radiolabeling yield of approximately 60%. The collected HPLC eluent was evaporated to dryness using the Biotage evaporator. No ²¹¹At activity was volatilized during evaporation. The product was re-dissolved in 400 μL PBS and analyzed by radio-HPLC to check identity (retention time) and radiochemical purity. The radio-HPLC chromatogram in FIG. 24 showed the collected product had high radiochemical purity.

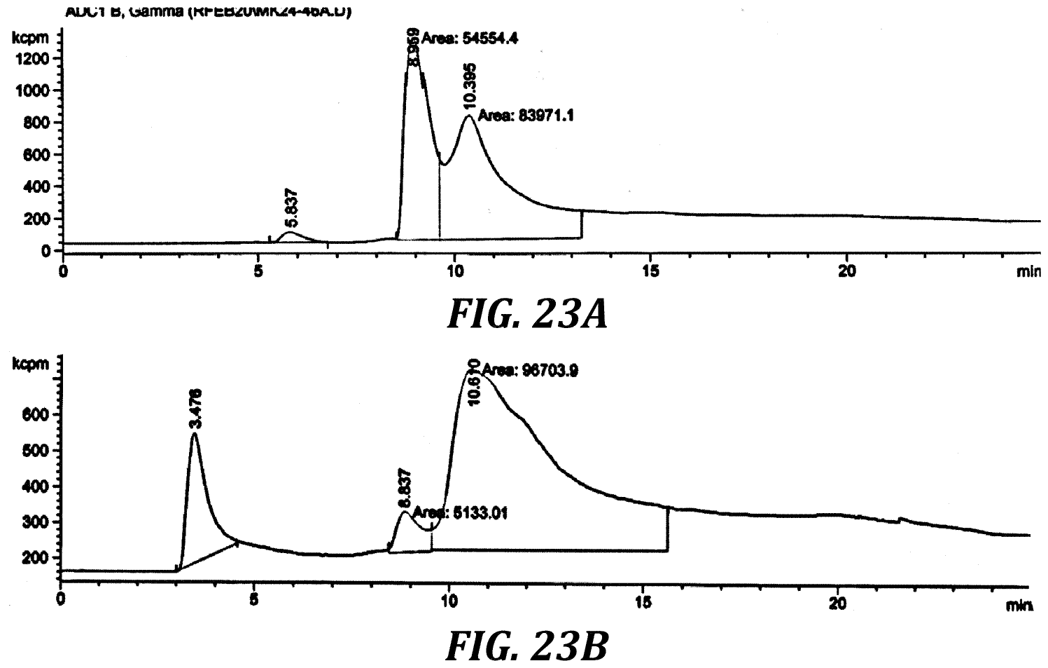

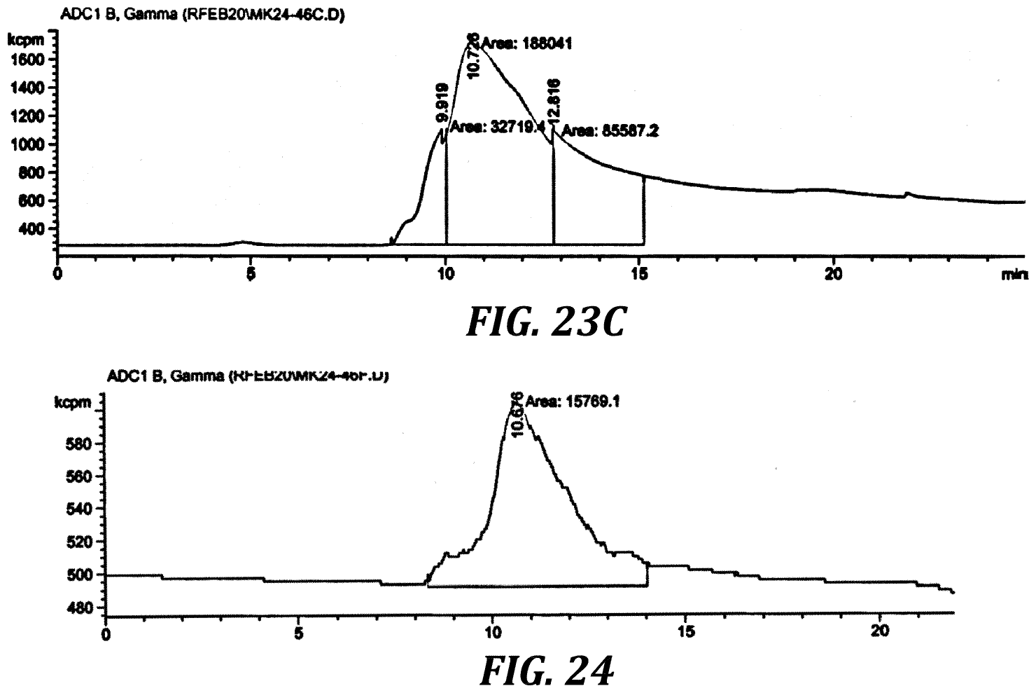

Reactions of closo-decaborate (2-) derivatives with ²¹¹At at room temperature are very rapid, being over almost immediately (less than 1 min). This fact suggests that the initial ²¹¹At-labeled product, 9a or 9b, may be represented in the peak at 9.0 min (FIG. 23A) and the astatonium ion 10a or 10b is likely represented in the radio-peak at 10.4 min.

Monoclonal antibodies (MAbs) conjugated with the At-reactive closo-decaborate (2-) bifunctional reagent (referred to as B10-NCS) have been problematic in that a large amount (e.g., 20-30%) of a higher molecular weight species if formed. Studies have shown that the high molecular weight species is likely a monoclonal antibody-B10 dimer and is formed by the addition of the oxidant chloramine-T (ChT). Conducting the ²¹¹At-labeling reaction without ChT alleviates the formation of the MAb dimer. The fact that the addition of an oxidant activates the ²¹¹At-MAb-B10 towards reaction helps explain the difference in tissue concentrations seen between some radioiodinated and astatinated MAb conjugates containing cleavable linkers. It has been shown that oxidases cause release of ²¹¹At from ary-At compounds. Oxidation of ²¹¹At that is bonded to an aromatic boron cage molecule (e.g., B10 moiety) does not release it as the B-At bond is stronger than the C-At bond. However, oxidation of aryboron-bonded ²¹¹At in cells by oxidases can make it reactive to cellular components.

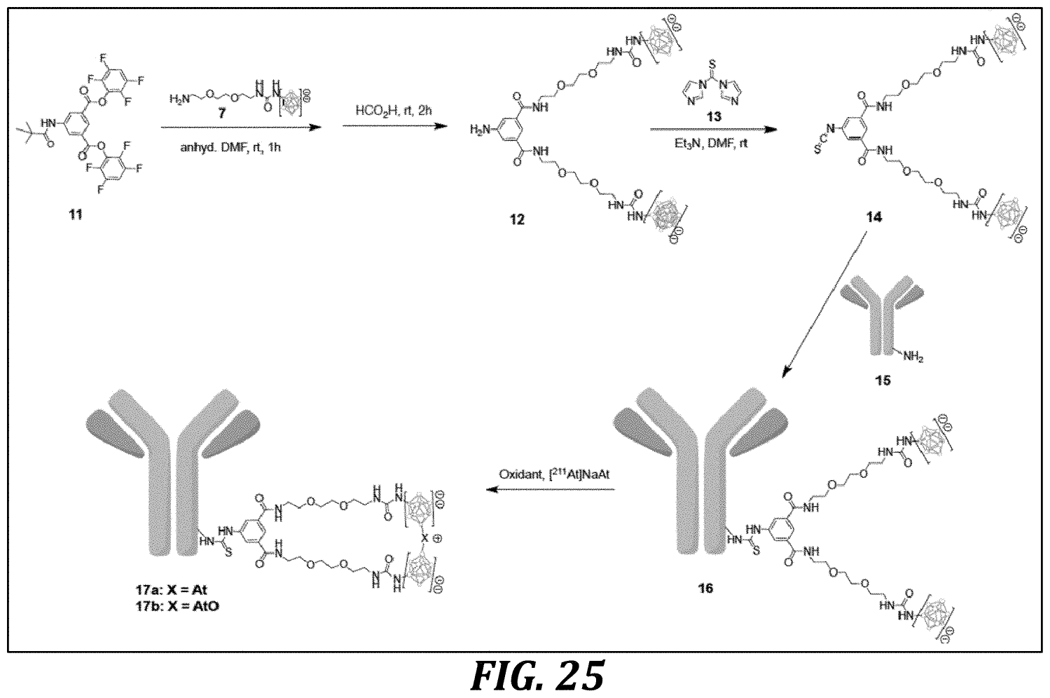

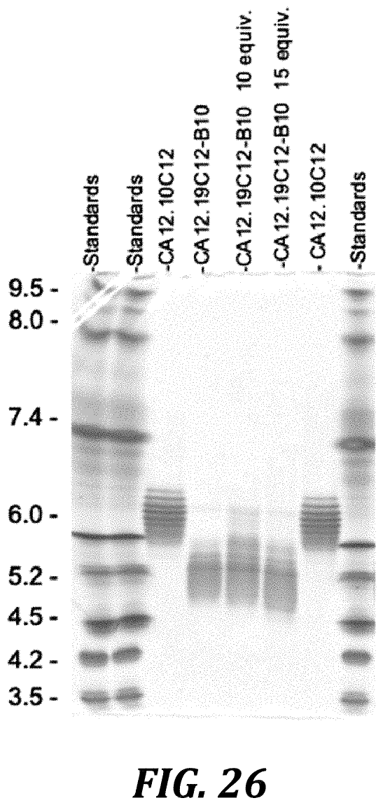

A study designed to demonstrate that formation of a B10-bridged astatonium ion alleviates the reactivity of the B10-At species was performed with a canine anti-CD45 MAb (CA12.10C12) conjugated with the bifunctional bis-B10 compound, 14. The synthesis of the bifunctional reagent 14 and its conjugation to a MAb is shown in FIG. 25 and described below. An isoelectric focusing (IEF) gel clearly indicated that the pI of the MAb changed to a lower pH as expected for addition of anionic conjugates. The gel shown in FIG. 26 shows the pIs of the (1) unaltered CA12.10C12 MAb, (2) CA12.10C12 conjugated to the mono-B10 bifunctional reagent (B10-NCS), (3 & 4) CA12.10C12 conjugated to the bis-B10, 14, after 10 or 15 equivalents of the reagent are reacted, relative to Novex IEF 3-10 precast gel Serva standards.

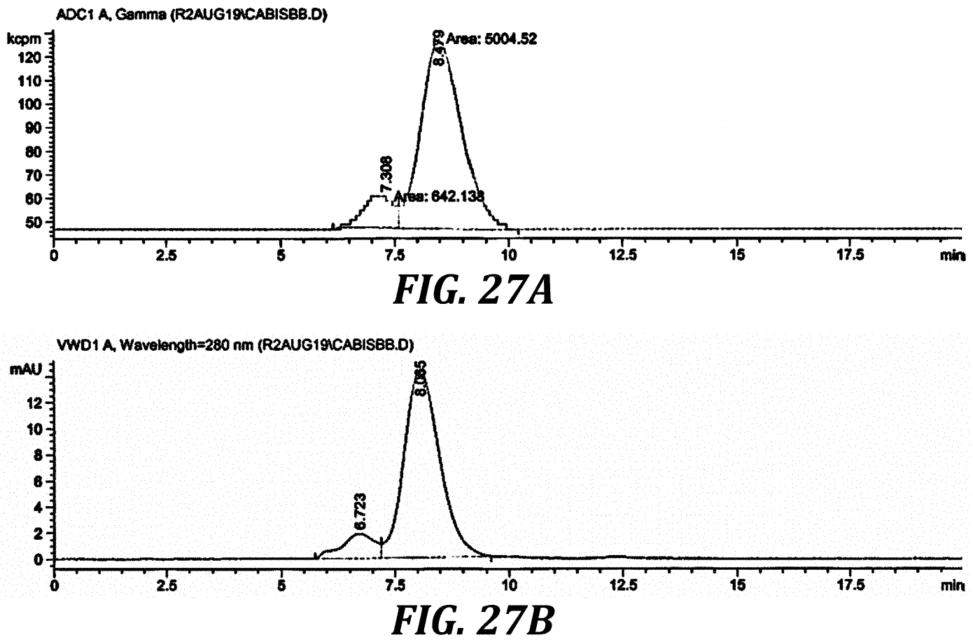

The MAb-bis-B10 [CA12.10C12-bis-B10], 16, prepared by reaction of CA12.10C12 with 15 equivalents of 14 was labeled with ²¹¹At using ChT as a chemical oxidant and the quantity of higher molecular weight species (by size-exclusion radio-HPLC) was obtained. Size-exclusion UV and radio-HPLC chromatograms of the product mixture are shown in FIGS. 27A and 27B. The higher molecular weight (HMW) peak at 6.7 min (UV) or 7.3 min (gamma) have similar area percentages to that of the unlabeled CA12.10C12-bis-B10 indicating that there was not increase in HMW species when using ChT as an oxidant.

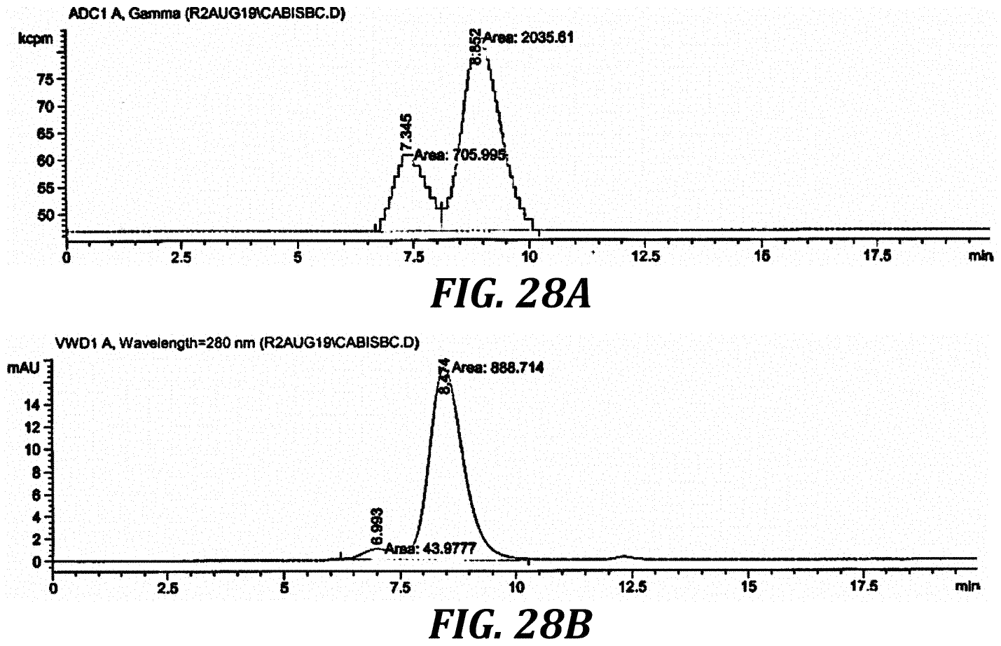

As a control, CA12.10C12 was conjugated with the bifunctional mono-B10 (B10-NCS) reagent and that conjugate, CA12.10C12-B10 was labeled with ²¹¹At using ChT as an oxidant. Size-exclusion UV and radio-HPLC chromatograms of the product mixture are shown in FIGS. 28A and 28B. The higher molecular weight (HMW) peak at 7.0 min (UV) or 8.9 min (gamma) have very different area percentages to that of the unlabeled CA12.10C12-B10 indicating that there was a significant increase (about 21%) in the ²¹¹At-labeled HMW species when using ChT as an oxidant. The results of this experiment clearly show that formation of the astatonium product, 17a or 17b, makes the ²¹¹At non-reactive to other MAb-bis-B10 conjugates in solution.

### Synthetic Procedures

The preparation of representative compounds and conjugates are described below and illustrated schematically in FIGS. 21 and 25.

Biotin-sarcosine adduct 1 and B10-NCO were synthesized as previously described. The structures for compounds 9 and 10 do not depict two compounds, rather they are shown as (a) At or (b) AtO derivatives.

3,5-CO₂Me-Ph-Sar-Biotin, 3. A solution of Biotin-Sar-OH (1, 1.0 g, 3.17 mmol), 3,5-CO₂Me-Ph-NH₂ (2, 0.603 g, 2.88 mmol), EDC (0.608 g, 3.17 mmol), DMAP (0.035 g, 0.288 mmol) and anhydrous DMF (15 mL) was stirred at room temperature for 16 h. H₂O (90 mL) and 1 N HCl (5 mL) was added to the reaction solution and stirred for additional 30 min. The precipitate was filtered, washed with water (500 mL), dried under vacuum for 16 h to give compound 3 as white solid. HPLC t_{R}=15.3 min. Yield 1.39 g (95%). LRMS (ES⁺) C₂₃H₃₁N₄O₇S (M+H)⁺, Calcd: 507.19, Found: 507.19.

3,5-CO₂H-Ph-Sar-Biotin, 4. A solution of 3,5-CO₂Me-Ph-Sar-Biotin (3, 1.0 g, 1.974 mmol), NaOH (0.197 g, 4.94 mmol) and 50% MeOH/H₂O (20 mL) was stirred at room temperature for 2 h. H₂O (90 mL) and 1 N HCl (6 mL) was added to the reaction solution and stirred for additional 30 min. The precipitate was filtered, washed with water (500 mL), dried under vacuum for 16 h to give compound 4 as white solid. HPLC t_{R}=12.0 min. Yield 0.86 g (91%). LRMS (ES⁺) C₂₁H₂₇N₄O₇S (M+H)⁺ Calcd: 479.16, Found: 479.16.

3,5-CO₂TFP-Ph-Sar-Biotin, 6. A solution of 3,5-CO₂H-Ph-Sar-Biotin (4, 0.406 g, 0.848 mmol), EDC (0.358 g, 1.867 mmol), TFP—OH, 5 (0.310 g, 1.867 mmol) and anhydrous DMF (10 mL) was stirred at room temperature for 16 h. 90 mL of H₂O was added to the reaction solution and stirred for additional 30 min. The precipitate was filtered, washed with water (500 mL), dried under vacuum to give compound 6 as white solid. HPLC t_{R}=16.7 min. Yield 0.62 g (94%). HRMS (ES⁺) C₃₃H₂₇F₈N₄O₇S (M+H)⁺ Calcd: 775.1473, Found: 775.1465.

[Et₃NH]₂ B₁₀H₉-dioxa-NH₂, 7. Compound 7 was prepared in two steps.

Step 1. A solution of [Et₃NH]₂ B₁₀H₉—N═C═O (prepared as described in Wilbur et al., Reagents for astatination of Biomolecules. 6. An intact antibody conjugated with a maleimido-closo-decaborate (2-) reagent via sulfhydryl groups had considerably higher kidney concentrations than the same antibody conjugated with an isothiocyanato-closo-decaborate (2-) reagent via lysine amines. Bioconjug. Chem. 2012; 23:409-420) (0.215 g, 0.591 mmol), BOC-dioxa-NH₂ (0.147 g, 0.591 mmol), NEt₃ (0.165 mL, 1.183 mmol), DMF (15 mL) was stirred and heated by microwave at 150° C. for 10 min. The crude solution was mixed with H₂O (2 mL) and purified via Biotage on an C18 FLASH 25+M column. The gradient mixture was composed of MeOH and 0.05 M triethylammonium acetate. Starting with 80% 0.05 M triethylammonium acetate, increased to 100% MeOH over the next 20 min). The crude product also can be purified by silica gel column (1.5 cm×25 cm) eluted with gradient solution from 100% EtOAc to 50% MeOH/EtOAc to provide 0.094 g of compound 12 (26%). HPLC t_{R}=9.8 min. HRMS (ES⁻) C₁₂H₃₄B₁₀N₃O₅(M+H)⁻ calcd: 410.3434, found: 410.3427.

Step 2. A solution of [Et₃NH]₂ B₁₀H₉-dioxa-NH—BOC (12, 94 mg, 0.154 mmol) and 96% formic acid (2 mL) was stirred at room temperature for 2 h. The reaction solution was evaporated under vacuum to dryness to yield 79 mg of compound 13 (100%). HPLC t_{R}=4.1 min. HRMS (ES⁻) C₇H₂₆B₁₀N₃O₃(M+H)⁻ Calcd: 310.2910, Found: 310.2912.

3,5-B₁₀H₉-dioxa-Ph-Sar-Biotin, 8. A solution of 3,5-CO₂TFP-Ph-Sar-Biotin, 6 (50 mg, 0.065 mmol), B₁₀H₉-dioxa-NH₂, 7 (79 mg, 0.155 mmol), NEt₃ (45 μL, 0.323 mmol) and anhydrous DMF (2 mL) was stirred at room temperature for 2 h. The solution was then triturated with EtOAc. The crude residue was dissolved in 50% MeOH/water and purified via Biotage on a C18 FLASH 25+M column. The gradient mixture was composed of MeOH and 0.05 M triethylammonium acetate. Starting with 80% 0.05 M triethylammonium acetate, increased to 100% MeOH over the next 15 min). Isolated yield of 8 was 44 mg (46%). HPLC t_{R}=7.6 min. LRMS (ES⁻) C₃₅H₇₂B₂₀N₁₀NaO₁₁S (M+Na)⁻ Calcd: 1082.7, Found: 1082.7.

²¹¹At-3,5-B₁₀H₉-dioxa-Ph-Sar-Biotin species, 9 and 10. To a solution containing 0.1 mg of 3,5-B₁₀H₉-dioxa-Ph-Sar-Biotin, 8 in 0.1 mL of a 5% HOAc/MeOH/H₂O solution was added to 1.63 mCi of [²¹¹At]NaAt in 0.1 mL of H₂O, pH 6-6.5. This mixture was reacted without addition of an oxidant (i.e., no chloramine-T) for 5 min at room temperature. Reversed-phase radio-HPLC showed 2 major peaks in nearly quantitative labeling at about 9 min and 10.4 min, which became one major peak when evaluated after 2 h 10 min (see FIG. 23C).

3,5-B₁₀H₉-dioxa-Ph-NH₂, 12. Compound 12 was prepared in two steps.

Step 1. A solution of 3,5-CO₂TFP-Ph-Boc, 11, (prepared as described in Hamblett et al. A streptavidin-biotin binding system that minimizes blocking by endogenous biotin. Bioconjug Chem. 2002; 13:588-598) (100 mg, 0.173 mmol), B₁₀H₉-dioxa-NH₂, 7 (195 mg, 0.381 mmol), NEt₃ (121 μL, 0.866 mmol) and anhydrous DMF (4 mL) was stirred at room temperature for one hour. The reaction solution was then evaporated to dryness under vacuum. The crude residue product was dissolved in 50% MeOH/water and purified via a Biotage C18 FLASH 25+M column. A gradient mixture composed of MeOH and 0.05 M triethylammonium acetate was used starting with 80% 0.05 M triethylammonium acetate, increased to 100% MeOH over the next 15 min. A 145 mg quantity (66%) of light-yellow solid, 3,5-B₁₀H₉-dioxa-Ph-Boc, was obtained. HPLC t_{R}=9.9 min.

Step 2. A solution of 3,5-B₁₀H₉-dioxa-Ph-Boc (100 mg, 0.079 mmol) and neat HCO₂H (2 mL) was stirred at room temperature for two hours. The reaction solution was then evaporated by a rotary evaporator under vacuum. The crude product was dissolved in 50% MeOH/water and purified via a Biotage C18 FLASH 25+M column. A gradient mixture composed of MeOH and 0.05 M triethylammonium acetate was used starting with 80% 0.05 M triethylammonium acetate, increased to 100% MeOH over the next 15 min. This yielded 71 mg (77%) of 12 as a light-yellow solid. HPLC t_{R}=7.0 min.

3,5-B₁₀H₉-dioxa-Ph-NCS, 14. A solution of 3,5-[Et₃NH]₂B₁₀H₉-dioxa-Ph-NH₂, 12 (50 mg, 0.043 mmol), 1,1′-thiocarbonyldiimidazole, 13 (9.91 mg, 0.056 mmol) and anhydrous DMF (2 mL) was stirred at room temperature for one hour. The reaction solution was then evaporated on a rotary evaporator under vacuum. The crude residue was dissolved in 50% MeOH/water and purified via Biotage C18 FLASH 25+M column. A gradient mixture composed of MeOH and 0.05 M triethylammonium acetate was used starting with 80% 0.05 M triethylammonium acetate, increased to 100% MeOH over the next 15 min. This provided 49.3 mg (95%) of 14 as a light-yellow solid. HPLC t_{R}=9.8 min. LRMS (ES⁻) C₂₃H₅₁B₂₀N₇NaO₈S (M+Na)⁻ Calcd: 828.5, Found: 828.5.

Conjugation of Bis-B10-NCS, 14, with CA12.10C12, 16. To 0.5 mL of 10.89 mg/mL CA12.10C12 MAb in PBS was added 0.5 mL 100 mM HEPES, 150 mM NaCl pH 8.6 followed by 10 or 15 eq (8.45/17 μL) of a 20 mg/mL bis-B10-NCS, 14, (mw=1169) in DMSO. The conjugation reaction was allowed to proceed overnight at room temperature with gentle tumbling. The MAb-bis-B10 conjugates, 16, was purified by SE-chromatography on PD-10 columns with collection in PBS. The protein fractions were combined and concentrated in a Vivaspin 20 (30K mwco). The fractions were concentrated and washed 5× to yield 0.7 mL of 7.0 mg/mL (90% protein recovery) in 1.0 mL of 4.8 mg/mL solution (88% protein recovery). IEF of the conjugates showed a shift in protein pI for both levels that is consistent with that of conjugations with mono-B10-NCS (FIG. 26).

²¹¹At-labeling of CA12.10C12-Bis-B10 (15 eq), 17a or 17b. A 100 μL quantity of 500 mM sodium phosphate, pH 6.8 was combined with 104 μL CA12.10C12-bis-B10, 16 (0.5 mg at 4.8 mg/mL). To this solution was added 0.1 mL of [²¹¹At]NaAt (1.15 mCi, pH 6.5-7) followed by 30 μL of 1 mg/mL solution of chloramine-T. After 5 min at room temperature, the reaction was quenched by adding 30 μL of a 1.0 mg/mL sodium metabisulfite solution in water. The mixture was passed over a PD-10 (G25) column and collected in PBS to provide a radiochemical yield of 62%. Size-exclusion radio-HPLC shows 2 peaks by gamma detection (7.3 and 8.5 min). The dimer at 7.3 min is about 10% (roughly equal to the dimer in the unlabeled conjugate).

²¹¹At-labeling of CA12.10C12-B10. A 100 μL quantity of 500 mM sodium phosphate, pH 6.8 was combined with 88 μL CA12.10C12-B10 (0.5 mg at 5.7 mg/mL). To this solution was added 0.1 mL of [²¹¹At]NaAt (1.28 mCi, pH 6.5-7) followed by 20 μL of 1 mg/mL solution of chloramine-T. After 5 min at room temperature, the reaction was quenched by adding 20 μL of a 1.0 mg/mL sodium metabisulfite solution in water. The mixture was passed over a PD-10 (G25) column and collected in PBS to provide a radiochemical yield of 50%. Size-exclusion radio-HPLC shows 2 peaks by gamma detection (7.3 and 8.8 min). The dimer at 7.3 min is about 26.5% (this peak is about 5% in the unlabeled conjugate).

While illustrative embodiments have been illustrated and described, it will be appreciated that various changes can be made therein without departing from the spirit and scope of the invention.

1. A compound represented by Formula (I): 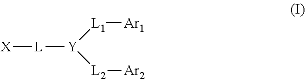
wherein
X is a group effective for associating the compound to a targeting agent;
L is a linker group that covalently links X to Y;
Y is trifunctional group that covalently links L, L₁, and L₂;
L₁ is a linker group that covalently links Y to Ar₁;
L₂ is a linker group that covalently links Y to Ar₂;
Ar₁ is an aromatic group that is reactive with an electropositive astatine species or is substituted with a group that makes it reactive with an electropositive astatine species and
Ar₂ is an aromatic group that is reactive with an electropositive astatine species or is substituted with a group that makes it reactive with an electropositive astatine species.

2. The compound of claim 1, wherein X is a functional group that is effective for covalently coupling the compound to a targeting agent or a ligand of a binding partner pair that is effective for binding the compound to a targeting agent.

3-7. (canceled)

8. The compound of claim 1, wherein the compound comprises two closo-decaborate (2-) moieties.

9. A compound represented by Formulae (IIA)-(IID): 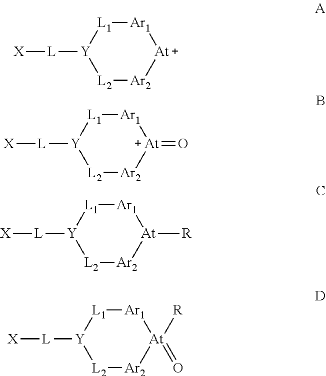
wherein
X is a group effective for associating the compound to a targeting agent;
L is a linker group that covalently links X to Y;
Y is trifunctional group that covalently links L, L₁, and L₂;
L₁ is a linker group that covalently links Y to Ar₁;
L₂ is a linker group that covalently links Y to Ar₂;
Ar₁ is an aromatic group that is reactive with an electropositive astatine species or is substituted with a group that makes it reactive with an electropositive astatine species and
Ar₂ is an aromatic group that is reactive with an electropositive astatine species or is substituted with a group that makes it reactive with an electropositive astatine species; and
R is a halide, hydroxyl, thiocyanate, isothiocyanate, or sulfhydryl, and
for structures A and C the astatine atom is in the +3 oxidation state and for structures B and D the astatine atom is in the +5 oxidation state.

10. (canceled)

11. A conjugate represented by Formulae (IVE)-(IVH): 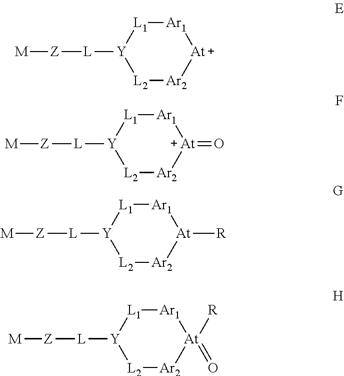
wherein
M is a targeting agent;
Z is a group formed from linking the targeting agent to L;
L is a linker group that covalently links Z to Y;
Y is trifunctional group that covalently links L, L₁, and L₂;
L₁ is a linker group that covalently links Y to Ar₁;
L₂ is a linker group that covalently links Y to Ar₂;
Ar₁ is an aromatic group that is reactive with an electropositive astatine species or is substituted with a group that makes it reactive with an electropositive astatine atom;
Ar₂ is an aromatic group that is reactive with an electropositive astatine species or is substituted with a group that makes it reactive with an electropositive astatine atom; and
R is a halide, hydroxyl, thiocyanate, isothiocyanate, or sulfhydryl, and
for structures E and G the astatine atom is in the +3 oxidation state and for structures F and H the astatine atom is in the +5 oxidation state.

12. The conjugate of claim 11, wherein M is a biomolecule.

13. The conjugate of claim 11, wherein M is a cancer-targeting biomolecule.

14. The conjugate of claim 11, wherein M is an antibody or a functional fragment thereof.

15-17. (canceled)

18. The compound of claim 1, wherein L₁ and L₂ are the same or different.

19. (canceled)

20. The compound of claim 1, wherein Y is a trifunctional aryl group or a trifunctional alkyl group.

21. The compound of claim 1, wherein Ar₁ is an anionic aromatic moiety or modified aromatic moiety that is reactive with an electropositive astatine species.

22. The compound of claim 1, wherein Ar₂ is an anionic aromatic moiety or modified aromatic moiety that is reactive with an electropositive astatine species.

23. The compound of claim 1, wherein Ar₁ and Ar₂ are independently selected from the group consisting of phenols, thiophenols, anilines, anisoles, organometallic substituted benzenes, organometallic substituted pyridines, nido-carboranes, monocarbon carboranes, and closo-borates (2-).

24. The compound of claim 1, wherein Ar₁ and Ar₂ are the same or different.

25. (canceled)

26. The compound of claim 1, wherein Ar₁ and Ar₂ are each closo-decaborate (2-).

27. (canceled)

28. A method for making an astatine compound, comprising reacting a compound of claim 1 with an electropositive astatine species.

29. The method of claim 28, wherein reacting the compound or conjugate with the electropositive astatine species does not include the use of an oxidant.

30. A method for introducing an astatine isotope into a subject, comprising administering a conjugate of claim 11 to a subject in need thereof.

31. A method for treating a disease or condition treatable by the administration of an astatine isotope, comprising administering a therapeutically effective amount of a conjugate of claim 11 to a subject in need thereof.
